(19) United States
(12) Patent Application Publication
(10) Pub. No.: US 2026/0099920 A1
(43) Pub. Date: Apr. 9, 2026

(54) VARIABLE COMPRESSION, DE-RESOLUTION, AND RESTORATION OF A MEDICAL IMAGE BASED UPON DIAGNOSTIC AND THERAPEUTIC RELEVANCE

(71) Applicant: Sean M. KELLY, Yorba Linda, CA (US)

(72) Inventor: Sean M. KELLY, Yorba Linda, CA (US)

(21) Appl. No.: 19/114,391

(22) PCT Filed: Sep. 25, 2023
(86) PCT No.: PCT/US2023/033644

§ 371 Date: Mar. 21, 2025

Related U.S. Application Data
(60) Provisional application No. 63/377,005, filed on Sep. 23, 2022.

(51) Int. Cl. G06T 7/00; G06N 3/0475; G06T 15/00; G16H 20/30; G16H 30/40; G16H 50/20; G16H 70/60

(52) U.S. Cl. CPC G06T 7/0012; G06N 3/0475; G06T 15/005; G16H 20/30; G16H 30/40; G16H 50/20; G16H 70/60; G06T 2207/30024

(57) ABSTRACT

An image processing platform obtains an input image of a specimen and globally down-resolves the input image into a down-resolved image. Subsequent to globally down-resolving the input image, the image processing platform concurrently globally up-resolves the down-resolved image into an up-resolved image using a generative adversarial network (GAN) model configured to reconstruct images including features corresponding to the specimen, and classifies a plurality of regions of the down-resolved image based on cellular morphologies and/or diagnostic relevance. The image processing platform conveys the up-resolved image to a communication network for delivery to a client device.

### RELATED APPLICATIONS

This application claims priority to U.S. Provisional Patent Application No. 63/377,005, entitled “Variable Compression, De-Resolution, and Restoration of a Medical Image Based Upon Diagnostic and Therapeutic Relevance” filed Sep. 23, 2022, which is hereby incorporated by reference in its entirety.

### TECHNICAL FIELD

This application relates to the field of digital pathology, namely the systems and methods for acquiring and processing electronic pathology slide images to include their acquisition, encoding, compression, storage, transmission, reconstruction, display, navigation, evaluation, diagnosis, and annotation into a resulting pathology report.

### BACKGROUND

In the field of pathology, mounted slides of pathology specimens may be converted into whole-slide digital images for subsequent use in computer-based diagnostic workflow. Examples of systems for performing such conversions are included in Bacus (U.S. Pat. No. 8,625,920) and Soenksen (U.S. Pat. No. 6,711,283).

But after twenty years of technical advancement and slow-but-gradual market adoption, most pathologists still use optical microscopes rather than a scanned slide image for their primary diagnostic workflow. One reason is that whole-slide digital images are very large, as high as 4.8 gigapixels. Equating this to a 4 k computer display with a resolution of 3840×2160 pixels, a typical whole-slide digital image could be expressed as roughly 500 full screens of detailed visual content. Such vast image files are generally sluggish to load and tedious to navigate compared to the instantaneous moving of a slide at one's fingertips. Pathologists generally prefer to actually touch the specimen slide with their own hands. It is a direct and profound connection to the patient, given the high stakes of the diagnostic outcome.

In addition to the problems associated with the capture and storage of whole-slide digital images, the diagnostic workflow involved in the viewing of such images can be inefficient and un-intuitive, when compared to the experience of viewing a physical specimen slide through a microscope. Current software solutions for digital diagnostic workflow typically follow the conventions of computerized user interface design such as click-boxes, radio buttons, scroll bars, and text boxes.

To improve the value proposition, many of these software-based solutions now include artificial intelligence (A.I.) to assist in the diagnostic process with recognition of cells, patterns, or features. But most pathologists have not yet chosen to entrust a third party software solution with such profoundly important matters. In addition, slide scanner instruments cost from $20 k to $300 k depending on their features and capacity. That is the better part of a year's income for most pathologists in the United States. A senior pathologist within ten years of retirement faces an ever diminishing return for such a costly reinvention of his or her established livelihood.

### SUMMARY

This disclosure solves the above needs by identifying, cataloguing, and spatially mapping known cellular, intracellular, and extracellular morphologies to assist the subsequent recreation and super-resolution of a high-resolution image. The approaches described herein further assess, quantify, and spatially map and define the regions of an original image to comprise two or more levels of diagnostic relevance. Such regions are then each assigned an optimal level of compression, generally inversely relative to diagnostic relevance. This avails a file-size advantage from the inherent fact that regular and normal cells are generally healthy and less diagnostically relevant in the diagnosis of one or more pathologies. This same aforementioned regularity and/or normalcy makes the healthy tissue regions and cellular morphologies more suitable for an A.I. assisted super-resolution circuit such a generative adversarial network (GAN). By designating a-priori a specialized GAN for each subtype of tissue or cell or state or morphology, the approaches described herein can more accurately reproduce an original high-resolution image from its down-resolved, pixel-shifted children.

An advantage of the approaches described herein is a reduced file-size for storage and transmission, as the high-resolution image may be recreated upon demand using the increasingly capable power of mobile phone processors and neural network software and firmware implementations. And whereas the total number of pixels to be stored is reduced, this advantage exists in addition to whatever compression is afforded by file formats such as JPEG-2000, whose lowest compression setting approximates lossless methods.

### BRIEF DESCRIPTION OF THE DRAWINGS

For a better understanding of the various described implementations, reference should be made to the Detailed Description below, in conjunction with the following drawings. Like reference numerals refer to corresponding parts throughout the drawings.

FIG. 1 is a diagram of an image processing environment configured to apply relevance-based variable compression, de-resolution, and restoration of a medical image in accordance with some implementations.

FIGS. 2A-2C are diagrams of a medical image acquisition process in accordance with some implementations.

FIG. 3 is a diagram of an alpha layer and metadata generation process in accordance with some implementations.

FIGS. 4A-4C are diagrams of a de-resolution, compression, and pre-validation process in accordance with some implementations.

FIG. 5 is a diagram of a super-resolution, decompression, and display process in accordance with some implementations.

FIG. 6 is a diagram of a pixel-shifting process in accordance with some implementations.

FIG. 7 is a flow chart of a process for applying relevance-based variable compression, de-resolution, and restoration of a medical image in accordance with some implementations.

FIGS. 8A-8B are diagrams of a system for displaying and interacting with medical images in accordance with some implementations.

FIGS. 9A-9D are diagrams of a device configured for interacting with medical images in accordance with some implementations.

FIGS. 10A-10B are diagrams of a device configured for interacting with medical images in accordance with some implementations.

FIG. 11 is a diagram depicting a plurality of usage modes of a device configured for interacting with medical images in accordance with some implementations.

FIGS. 12A-12C are diagrams depicting usage modes of a device configured for interacting with medical images in accordance with some implementations.

FIG. 13 is a diagram of a system for collaborative interaction with medical images in accordance with some implementations.

FIG. 14 is a diagram of a system for interacting with medical images using facial gestures in accordance with some implementations.

FIG. 15 is a diagram of a system for interacting with medical images using facial gestures in accordance with some implementations.

FIG. 16 is a diagram of a system for interacting with medical images using facial gestures in accordance with some implementations.

### DETAILED DESCRIPTION

The present disclosure describes systems and methods for using machine vision and A.I. for better image compression, transmission, super-resolved rendering, and subsequent diagnostic workflow with regards to medical images. By pre-identifying, mapping and indexing the diagnostically relevant tissue features and image regions against a library of known cells, tissue types and features, the systems and methods described herein enable faithful subsequent reconstruction of the original compressed image, and also swifter navigation of the image during diagnostic workflow. Using one or more peripheral controlling devices, the systems and methods described herein use a diagnostic feature index to snap each successive diagnostically relevant tissue feature or image region into the pathologist's focus of attention.

The present disclosure aims to isolate and extract diagnostically relevant (i.e., suspect and/or potentially cancerous) cells, tissues, and image regions, affording them a less-lossy compression than the remaining, less diagnostically relevant cells, tissues, and image regions, which are, by definition, more normal and regular in their states, attributes, and morphology, both individually, and in the aggregate. Such normal and regular image content are therefore more suitable for higher compression levels of various types such as wavelet, token library such as LZW, color compression, pixel-shift super-resolution, run-length encoding, and others.

In general, the less relevant cells and tissue features will be more normal or regular and therefore more predictable and suitable for advantageous de-resolution and compression, while the more relevant cells and tissues may be extracted and preserved in their original, or close to original, resolution with minimal to no compression applied in order to ensure the highest level of accuracy for those image regions that are most important for diagnosing medical issues and determining therapeutic courses of action. By compressing less relevant image regions and preserving more relevant image regions, the systems and methods described herein optimally balance the trade-off between more workable image file sizes and pathologist trust.

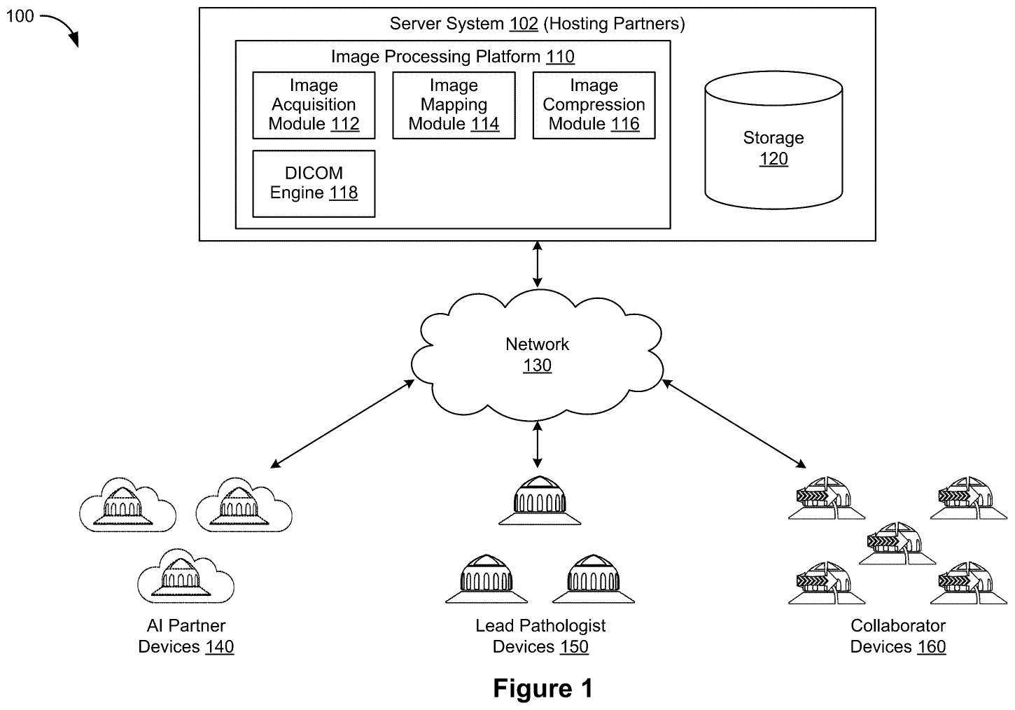

FIG. 1 is a diagram of an image processing environment 100 configured to apply relevance-based variable compression, de-resolution, and restoration of a medical image in accordance with some implementations.

Specifically, environment 100 illustrates an electronic network 130 that may be connected to a server system 102, including hosting partners such as hospitals, laboratories, and/or doctors' offices, and so forth. For example, physician servers, hospital servers, clinical trial servers, research lab servers, and/or laboratory information systems, may each be connected to an electronic network 130, such as the Internet, through one or more computers, servers, and/or handheld mobile devices. Server system 102 include processing devices that are configured to implement an image processing platform 110, which includes an image acquisition module 112, an image mapping/classifier module 114, an image compression module 116, and a DICOM compliance engine 118, each discussed in more detail below.

Environment 100 facilitates efficient and trustworthy remote access to medical images among pathologist devices 150 and collaborator devices 160. Devices 150/160 are electronic devices, sometimes referred to as client devices, associated with respective users. Devices 150/160 may include, but are not limited to, smartphones, tablet computers, laptop computers, desktop computers, smart cards, voice assistant devices, or other technology (e.g., a hardware-software combination) known or yet to be discovered that has structure and/or capabilities similar to mobile devices or computer peripherals as described herein. In some implementations, devices 150/160 may include peripheral devices, such as dials configured to navigate regions of the medical images, features of which are disclosed in greater detail below. Devices 150/160 are communicatively coupled to server system 102 using a communication capability (e.g., modem, transceiver, radio, and so forth) for communicating through the network 130. AI partner devices 140 may approximate functionality of devices 150/160 or otherwise assist in certain aspects of image analysis as described in more detail below.

Server system 102 is communicatively coupled to devices 140-160 by one or more communication networks 130. The communication network(s) 130 are configured to convey communications (messages, signals, transmissions, and so forth). The communications include various types of information and/or instructions including, but not limited to, data, commands, bits, symbols, voltages, currents, electromagnetic waves, magnetic fields or particles, optical fields or particles, and/or any combination thereof. The communication network(s) 130 use one or more communication protocols, such as any of Wi-Fi, Bluetooth, Bluetooth Low Energy (BLE), near-field communication (NFC), ultra-wideband (UWB), radio frequency identification (RFID), infrared wireless, induction wireless, ZigBee, Z-Wave, 6LoWPAN, Thread, 4G, 5G, and the like. Such protocols may be used to send and receive the communications using one or more transmitters, receivers, or transceivers. For example, hard-wired communications (e.g., wired serial communications) may use technology appropriate for hard-wired communications, short-range communications (e.g., Bluetooth) may use technology appropriate for close communications, and long-range communications (e.g., GSM, CDMA, Wi-Fi, wide area network (WAN), local area network (LAN), or the like) may use technology appropriate for remote communications over a distance (e.g., over the Internet). In general, the communication network(s) 130 may include or otherwise use any wired or wireless communication technology that is known or yet to be discovered.

Server system 102 (e.g., including one or more hospital servers, clinical trial servers, research lab servers, and/or laboratory information systems) may create or otherwise obtain images of one or more patients' cytology specimens, histopathology specimens, slides of the cytology specimens, digitized images of the slides of the histopathology specimens, or any combination thereof. Server system 102 may also obtain any combination of patient-specific information, such as age, medical history, cancer treatment history, family history, past biopsy or cytology information, etc. Server system 102 processes digitized slide images and transmits the processed images to devices 150/160 over network 130. Server system 102 may include one or more storage devices 120 for storing the aforementioned images and processed image data. Server system 102 may also include processing devices for processing the images and data stored in the storage devices 120, such as one or more processors, each including one or more processing cores. Server system 102 may further include one or more machine learning tools or capabilities, features of which are described in more detail below. Additionally or alternatively, the present disclosure (or portions of the system and methods of the present disclosure) may be performed on a local processing device (e.g., a laptop).

Server system 102 may be implemented on one or more standalone data processing apparatuses or a distributed network of computers. In some implementations, server system 102 also employs various virtual devices and/or services of third party service providers (e.g., third-party cloud service providers) to provide the underlying computing resources and/or infrastructure resources of the server system 102. In some implementations, server system 102 includes, but is not limited to, a handheld computer, a tablet computer, a laptop computer, a desktop computer, or a combination of any two or more of these data processing devices or other data processing devices.

Storage 120 includes a non-transitory computer readable storage medium, such as volatile memory (e.g., one or more random access memory devices) and/or non-volatile memory (e.g., one or more flash memory devices, magnetic disk storage devices, optical disk storage devices, or other non-volatile solid state storage devices). The memory may include one or more storage devices remotely located from the processor(s). The memory stores programs (described herein as modules and corresponding to sets of instructions) that, when executed by the processor(s), cause the server system 102 to perform functions as described herein. The modules (e.g., 112-118) and data described herein need not be implemented as separate programs, procedures, modules, or data structures. Thus, various subsets of these modules and data may be combined or otherwise rearranged in various implementations

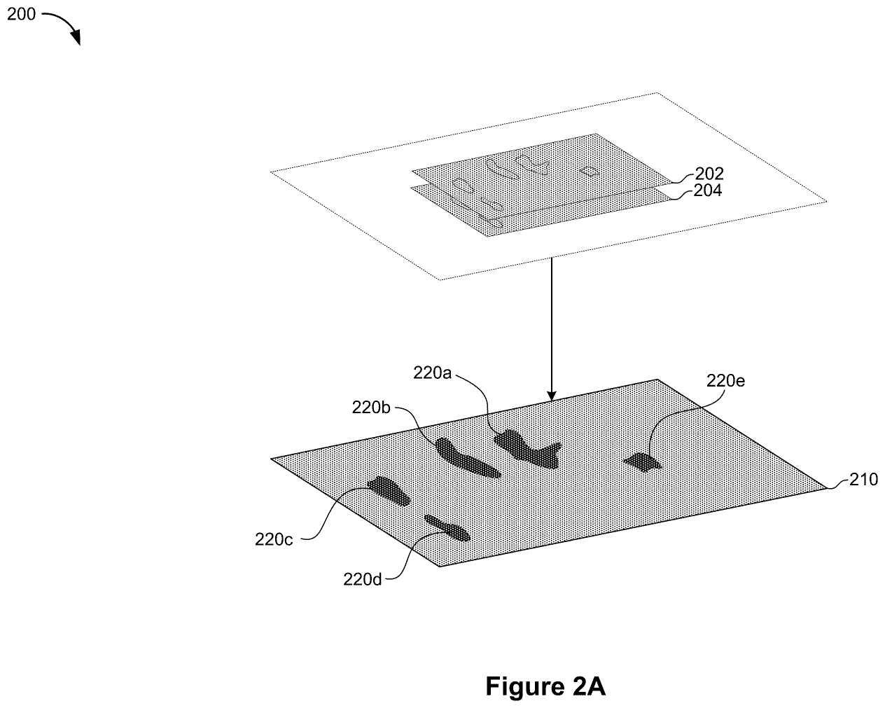

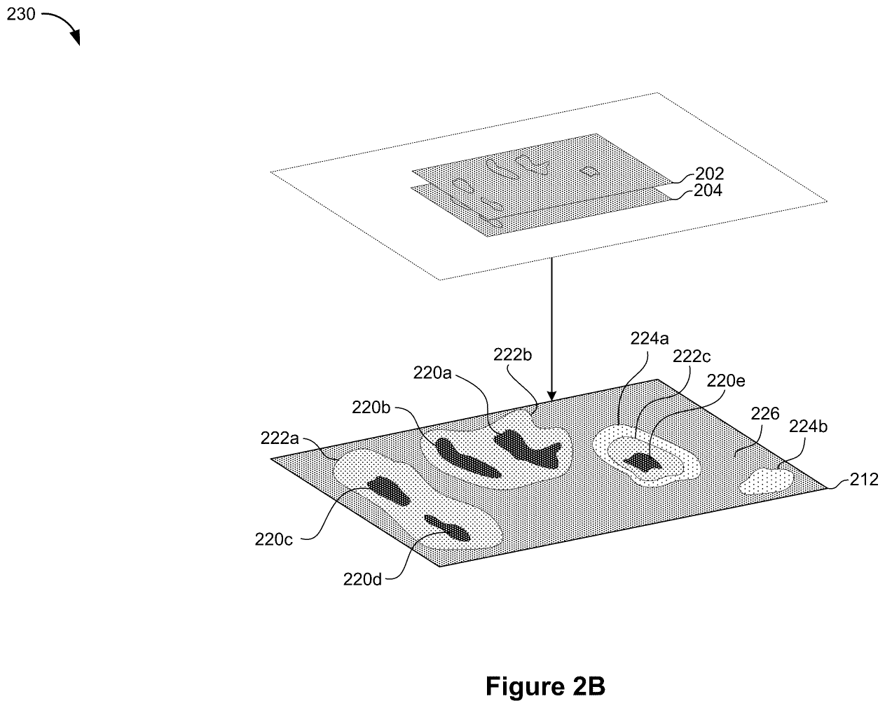

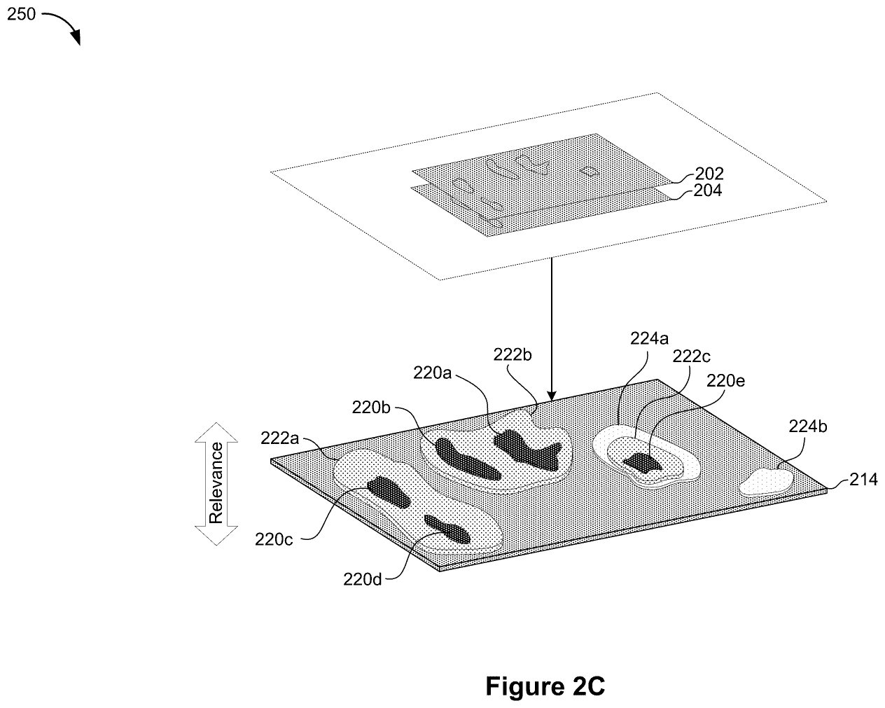

FIG. 2A is a diagram of a medical image acquisition process 200 in accordance with some implementations. In some implementations, one or more source images 202, 204 are scanned at a resolution corresponding to a capability of a scanning camera that is used to scan the source images (referred to as a native scanning resolution). While the figure shows two images 202 and 204, other implementations may include one image or more than two images (e.g., 3 images, 4 images, and so forth). For implementations in which a plurality of images are scanned, the plurality of images may be pixel-shifted images. Specifically, each successive image may be offset by at least one pixel in at least one direction. This may dramatically reduce scan times, and could cut in half the cost of the scanner camera.

Source images 202 and 204 may be obtained by image acquisition module 112 of the image processing platform 110 (FIG. 1). That is, the source images may be obtained locally at a hospital at which server system 102 is located, or remotely from which server system 102 is located.

Each pixel-shifted source image 202, 204 is a component of a final source image 210. Stated another way, the pixel-shifted source images 202, 204 are combined into a final source image 210. In some implementations, the final source image 210 could not have been incident upon an image sensor of a scanning camera because it is either a composited image comprised of the optimum pixel(s) from within a non-planar volumetric z-stack or a stitched series of tiles which is beyond the resolution of the original image sensor.

The final source image 210 includes one or more regions (e.g., regions 220a-220e), generally referred to as areas of interest, or more specifically referred to as diagnostically relevant regions or therapeutically relevant regions. Such regions are diagnostically relevant if they include, for example, a cell morphology that is relevant to the diagnosis of a patient associated with the tissue in the image 210. Likewise, such regions are therapeutically relevant if they include, for example, a feature that is relevant to a therapeutic outcome of a patient associated with the tissue in the image 210. Throughout this disclosure, the “diagnostic relevance” may refer to diagnostic relevance and/or therapeutic relevance.

In some implementations, therapeutic relevance may include such factors that may reasonably serve to guide selection of treatment, suitability for various drug trials or research programs, optimum specific venue, or institution for treatment. Stated another way, therapeutic relevance corresponds to the matching of a specimen/patient with studies, drug trials, and/or treatment regimes.

In some implementations, diagnostic relevance is not merely limited to that which indicates the decisive presence or likelihood of cancer, dysplasia, fibrosis, inflammation, or any other pathology, but also any feature within a specimen which relates to the positive or negative determination of pathogenesis, e.g., the presence or state of a cancer, or the degree of remission or minimum residual disease, or the degree and location of dysplasia, or the degree and composition of fibrosis or inflammation, whether such an indicator is in the state or morphology of organelles or cells or tissues or protein matrices or fluids or organic polymers or anatomic structures or in the secondary evidence brought about through fixation, clearing, processing, sectioning, mounting, staining, and any other method practiced within a histology laboratory. An example list of relevant features that image mapping/classifier module 114 may index in an AI scanner includes cells, nuclei, organelles, lacunae, glands, blood vessels, signet ring cells, atypical pleomorphism, and atypical collagen structure.

In some implementations, diagnostic and/or therapeutic relevance may be based on the quantifying of a number of instances of a thing or cell or state within a particular inter-proximity or region. For example, diagnostic and/or therapeutic relevance may be based on a how many of cells of a particular type are within a certain proximity to one another or contained with a field of a defined size. For example, diagnostic and/or therapeutic relevance may be based on the counting of mitotic figures within a specific area. As another example, in breast cancer, one of the criteria for grading is the number of mitotic figures in the tumor in ten high-power (40×) fields. A 40× field can also be considered an area if the specifications of the microscope are known. Melanoma uses the number of mitoses per square mm. Counting mitoses can be important in grading a malignant tumor and help distinguish benign and malignant tumors. Gastrointestinal stromal tumors and many other sarcomas/soft tissue tumors are stratified into benign, uncertain malignant potential, and malignant based on the number of mitotic figures in many high power fields, often 50 or more.

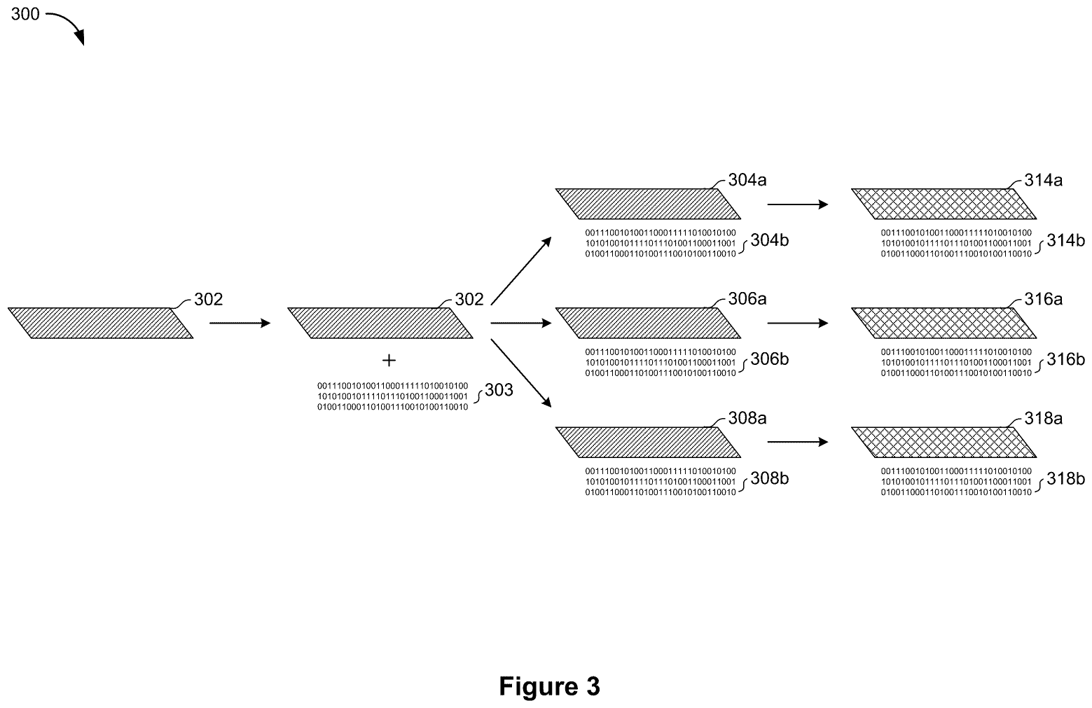

In some implementations, relevance regions (also referred to as zones, subsets, or portions) can be based upon the level of relevance, the type or context of relevance, or any combination thereof, in a plurality of layers as discussed in more detail below (e.g., with reference to FIG. 3).

FIG. 2B is a diagram of a medical image acquisition process 230 in accordance with some implementations. Acquisition and processing of the source images 202 and 204 are similar to that described above with reference to FIG. 2A. In process 230, however, the final source image 212 includes a plurality of levels of areas of interest, or levels of diagnostic and/or therapeutic relevance. For example, regions 220a-220e have a first level of diagnostic and/or therapeutic relevance, regions 222a-222c have a second level of diagnostic and/or therapeutic relevance lower than the first level, and regions 224a-224b have a third level of diagnostic and/or therapeutic relevance lower than the second level. Regions of the final source image 212 not included in regions 220a-220e, 222a-222c, and 224a-224b (e.g., region 226) have a level of diagnostic and/or therapeutic relevance lower than the third level. Regions having the highest level of relevance (e.g., 220a) may be the most beneficial to a pathologist in diagnosing a patient or determining a therapeutic course of action, while regions having the lowest level of relevance (e.g., 226) may be the least beneficial (or have no benefit) to a pathologist in diagnosing a patient or determining a therapeutic course of action.

FIG. 2C is a diagram of a medical image acquisition process 250 in accordance with some implementations. Acquisition and processing of the source images 202 and 204 are similar to that described above with reference to FIGS. 2A-2B. In process 250, however, the final source image may be visualized in a three dimensional format, with regions separated in the z-axis by their respective levels of diagnostic and/or therapeutic relevance. As such, the visualization in FIG. 2C of the final source image may resemble a topographic map, with relevance levels of the plurality of regions of the image signified as elevation. With such a visualization, a three-dimensional fly-through model of the specimen in the final source image may be displayed with labels, snap-to points, and/or tiers of relevance being shown as the various z-elevations. In some implementations, the most relevant pixels for a given (x, y) location in the image may be identified in a volumetric z-stack.

In some implementations, image mapping/classifier module 114 uses a third-party input aggregator (TPI or TPIA) to identify and map the regions of relevance in the final source image. In some implementations, image mapping/classifier module 114 uses relevance ratings provided by in-house systems or personnel to identify and map the regions of relevance in the final source image.

In some implementations, diagnostic and/or therapeutic relevance can be a composite from a plurality of diagnostic and/or therapeutic relevance ratings provided from several different sources.

In some implementations, in the analysis of a composite z-stack, the image mapping/classifier module 114 assesses diagnostic and/or therapeutic relevance independent of focal clarity. A feature or image region may be highly diagnostically and/or therapeutically relevant yet out of focus or less-sharply in focus compared with a feature above or below it in the z-axis. Some systems that use selective processes within a z-stack generally may select best focus, rather than diagnostic relevance, which is a different attribute entirely. However, the disclosed system could, for example, create a composite image with defocused features which could be subsequently sharpened by an algorithm or neural network, specifically because they were first determined and selected on the basis of diagnostic and/or therapeutic relevance.

In some implementations, the relevance ratings of the specimen in the final source image features and/or image regions (e.g., regions 220, 222, and 224) may be organized into hierarchical classes, categories, or associated metatags.

In some implementations, direct a-priori data (e.g., pre-designated spot-locations or regions of the slide or features of the specimen that the pathologist or oncologist find important) and metadata (e.g., health information about the patient, or the community/environment in which the patient lives or works or conducts commerce) may serve as inputs for a machine vision/learning system in image mapping/classifier module 114 to assess and index specimen features and score diagnostic and/or therapeutic relevance of the various regions in the final source image.

In some implementations, image mapping/classifier module 114 may accommodate revisions by pathologists to the relevance ratings assigned to the image regions. In some implementations, such revisions may track an individual relevance model for the pathologist. In some implementations, such revisions may inform the continuous improvement of a global model for determining relevance of a particular cell morphology or other feature of a medical image. A plurality of such profiles for modelling and ever-improving concordance on each level may be based on self-concordance of a pathologist, concordance within a multi-physician pathology practice, and/or concordance to a board standard. In some implementations, there may be a systemic shifting of any or all of the above towards better sensitivity and specificity (thus advancing the field).

In some implementations, image mapping/classifier module 114 generates a cellular index of the specimen in the source image, and saves this cellular index as metadata corresponding to the image. The data in the cellular index may be mapped as vector files associated with coordinate values of the image at which certain features in the cellular index are present. The metadata may be included in one or more discreet files included in a file wrapper or may be included in a file header of the image file, or may be steganographically included in the image file.

The cellular index is a precise and complete, descriptive and instructive, quantitate and qualitative assessment of the biopsy specimen in the source image. The cellular index may include intracellular, extracellular, qualitative, and/or quantitative attributes of the specimen. The types of specimen may include any of bone, blood, fluids, tissue, and so forth. The cellular index itself may be any of an index, census, compendium, map, list, and so forth. The cellular index may be referred to as a cellular index and compression key (CICK). In some implementations, the cellular index may include pre-annotations.

An example list of cellular index features (indexable features of the specimen) included in the metadata for a given medical image includes any of Abscess, Absorptive cells (enterocytes), Acid fast bacillus/bacilli, Acinar (alveolar) glands, Acinus/Acini, Adipocyte, Adipose tissue, Adventitia, Alveolar space, Alveoli, Amacrine cells, Ameloblast, Apocrine cell, Arrector pili, Arteriole, Artery, Astrocyte, Atherosclerotic plaque, Atypical mitosis/mitoses, Bacillus/Bacilli, Bacterium/Bacteria, Band, Barr body, Basal lamina, Basophil, Basophilia, Basophilic stippling, Bile duct, Blast, Blood vessels, Bone, Bone marrow, Bowmans capsule, Brunner's glands, Brush border, Bunina bodies, Cabot rings, Calcification, Canaliculi, Cancellous bone, Capillary, Capsule, Cardiac muscle, Cardiac valve, Cartilage, Cells, Cementum, Central vein, Centriole, Chief cells, Chondroblast, Chondrocyte, Chromatin, Cilium/Cilia, Collagen, Columnar cell, Connective tissue, Coprus albicans, Cords, Cords of Bilroth, Cornea, Corona radiata, Corpus luteum, Cortex, Cowdry bodies, Crypts, Crypts of Lieberkuhn, Cyst, Cytoplasmic vacuole, Decidual cell, Dentin, Dermis, Descemet's membrane, Duct, Ductal epithelium, Dutcher Bodies, Dystrophic calcification, Eccrine duct, Eccrine gland, Elastic fiber, Enamel, Endocardium, Endocrine cell, Endometrial glands, Endometriosis, Endomysium, Endosteum, Endothelial cell, Endothelial cells, Enteroendocrine cells, Eosinophil, Eosinophilia, Ependymal cell, Epidermis, Epineurium, Epithelium, Erythrocyte (red blood cell), Exocrine cell, External elastic lamina, Fascia, Fasicle, Fenestrated endothelium, Fibrin, Fibriopurulent exudate, Fibroblasts, Fibroid, Fibrosis, Fibrovascular core, Fimbriae, Follicle, Fruiting body, Fungal hyphus/hyphae, Fungal yeast, Fungus, Ganglion cell, Gangrenous necrosis, Gastric pits, Germinal center, Giant cell, Glands, Glial cells, Glomerulus/glomeruli, Goblet cells, Graafian follcile, Granule, Granulocyte, Granuloma, Granulosa cell, Ground substance, Hair, Hair follicle, Halo cells, Hassall's corpuscle, Haversian canal, Heinz bodies, Helicobacter/Helicobacter pylori/H. pylori, Helicobacter-like organism, Hematogone, Hemosiderin, Hepatocytes, Hilum, Histiocyte, Hofbauer cell, Howell-Jolly bodies, Hyaline cartilage, Hydroxyapatite, Hyperchromasia, Hyphus/Hyphae, Hypnozoites, Immunoblast, Inclusion bodies, Inner circular muscle, Internal elastic lamina, Interstitial cells of Cajal, Interstitial cells of Leydig, Intranuclear inclusions, Islet cell, Islets of Langerhans, Juxtaglomerular cells, Keratin, Keratin pearl, Keratinocyte, Kupffer cells (liver macrophages), Lacteals, Lacunae, Lamina propria, Langerhans cell, Lens, Leukocyte (white blood cell), Lewy bodies, Leydig cell, Ligament, Lipofuscin, Loose connective tissue, Lumen, Luminal contour, Luminal space, Luschka's ducts, Lymphatics, Lymphoblast, Lymphocytes, Lymphoid follcile, Lysosome, Macrophages, Macula adherens, Macula densa, Mast cell, Megakaryocyte, Meiosis, Melanin, Membrane, Merkel cell, Mesangial cells, Mesenchyme, Mesothelial cell, Mesothelium, Metamyelocyte, Michalis-Gutmann body, Microcalcification, Microorganism, Microvilli, Mitosis/Mitoses, Mitotic figure, Molluscum bodies, Monoblast, Monocyte, Mucosa, Mucous cells, Muscle tissue, Muscularis externa, Muscularis mucosae, Myeloblast, Myelocyte, Myenteric (Auerbach's) plexus, Nabothian cyst, Necrosis, Nerve, Nervous tissue, Neuron, Neutrophil, Nissl substance, Nuclear membrane, Nucleolus/Nucleoli, Nucleus/nuclei, Oligodendrocyte, Oocyte, Ordinary connective tissue, Organelles, Osteoblast, Osteoclast, Osteocyte, Osteoid, Outer longitudinal muscle, Ova, Oxyntic cell, Oxyphilic cell, Pacinian corpuscle, Pancreatic acinar cells, Paneth cells, Papilla/Papillae, Papillary dermis, Pappenheimer bodies, Parasite, Parasympathetic ganglion cells, Parenchyma, Parietal (oxyntic) cells, Periarterial lymphatic sheath, Pericyte, Perikaryon, Periosteum, Peyer's patches, Phagocyte, Phagocytosis, Pick bodies, Pigment, Plasma cell, Plasma membrane, Plasmodium species, Platelet, Pleomorphism, Pneumocyte, Podocytes, Polychromatic erythroblast, Portal triad, Proerythroblast, Promyelocyte, Psammoma body, Pulp, Purkinje cell, Purkinje fibers, Pyramidal cell, Red pulp, Reed-Sternberg cell, Reinke crystals, Respiratory epithelium, Rete ovary, Rete peg, Rete ridges, Rete testis, Reticular dermis, Reticulocyte, Retinal pigmented epithelium, Russell bodies, Sarcolemma, Schistocytes, Sebaceous gland, Secretory epithelium, Seminal vesicle, Seminiferous tubules, Seroma, Serosa, Serous cells, Sertoli cell, Signet ring cells, Simple columnar epithelium, Simple cuboidal epithelium, Simple squamous epithelium, Sinus/sinusoid, Sinusoids, Skeletal muscle, Smooth muscle cells, Smudge cell, Spermatid, Spermatocyte, Spermatogone, Spermatogonia, Spermatozoa, Spindle cell, Spirochete, Sporozoites, Stratified squamous epithelium, Stroma, Subcapsular space, Subcutaneous adipose tissue, Submucosa, Submucosal (Meissner's) plexus, Submucosal glands, Surface mucous cells, Sweat gland, Synovium, Tendon, Terminal bar, Thrombus, Thymocyte, Treponemes, Tubular glands, Tubule, Urothelium, Vacuole, Vascular plexus, Vein, Venule, Villus/villi, Viral inclusion, Warthin-Finkeldey bodies, Whartons jelly, white pulp, Wolffian duct, Woven bone, and Yeast. This list is provided for illustrative purposes and is not meant to be exhaustive or limit the scope of this disclosure in any way.

In some implementations, other data related to the above features may also be indexable, such as attributes and/or parameters of any of the aforementioned features including but-not-limited-to width, height, thickness, diameter, optical density, chromatic bias, opacity, polarization, dimensional distortion from normalcy, angular bias, rotational state, sharpness of focus, and so forth.

In some implementations, other types of features may also be indexable. For example, confounding or distorting features may be indexable, such as cracked slide, cracked cover slip, excess cover-slip medium, entrapped bubble, smudge, finger-print, protein speck inclusion, hair inclusion, folded tissue edge, tissue wrinkle, non-recurring tissue thickness variation, recurring tissue-thickness variation, dried top residue, dried bottom residue, misplaced label, separated cover-slip, extra slide-glass. While these features may be associated with the medical image, they are not associated with the specimen itself. As such, in some implementations, these features may be logged but not included in the cellular index.

Any subset of the aforementioned features in the cellular index may be generated as a vector file or spline, which may describes an area that encloses a plurality (e.g., thousands) of cells in an efficient manner. As such, in some implementations, an example ratio of pixels versus logged features may be at least 1,000 to 1. In some implementations, the cellular index may include coincident metatags, as some features can be more than one thing (e.g., a skin cell may be part of the wall of a gland). As most normal/healthy tissues will contain dozens or hundreds of similar cells, the index may be run-length-encoded, such that the net ratio should still come back down well below 1,000 to 1. And since at least half of the index may be unused for any given sample, image mapping/classifier module 114 may use a serialized per-specimen roster, which can potential decrease the size of entries in the index to an eight bit (one-byte) identifier. As such, the per-specimen vocabular can potentially be limited to well below 256 feature types.

FIG. 3 is a diagram of an alpha layer and metadata generation process 300 in accordance with some implementations. In some implementations, tiered levels of diagnostic relevance are established through machine vision analysis of the system (implemented by image mapping/classifier module 114), each layer comprising one or more regions of the total image, hence a fractional percentage of the total pixels. Such regions may then be separated into discrete alpha-layers, each comprising a tier of diagnostic relevance. These alpha layer images may then be compressed using the optimal compression type for their composition, and with the compression level inversely related to the diagnostic relevance.

For example, in process 300, a medical image 302 (e.g., corresponding to image 214, FIG. 2C) is obtained by image acquisition module 112 and mapped by image mapping/classifier module 114. As a result of the mapping, a metadata layer 303 corresponding to the image layer 302 is generated, including a cellular index including diagnostic relevance scores by region and by feature. This metadata layer may also include a recommended sequential diagnostic workflow (explained in more detail below), including pre-annotation, or a composite such recommendation aggregated from multiple diagnostic sources.

The image 302 and cellular index 303 are split into three distinct alpha layers, 304, 306, and 308, each defined for an optimally selected type, format, ratio, and/or degree of compression versus image fidelity. For example, layer 304 (including image file or layer 304a and metadata file or layer 304b) includes the most diagnostically relevant portions of the image (e.g., 220a-220e, FIG. 2C), layer 306 (including image file or layer 306a and metadata file or layer 306b) includes moderately diagnostically relevant portions of the image (e.g., 222a-222c, FIG. 2C), and layer 308 (including image file or layer 308a and metadata file or layer 308b) includes the lesser diagnostically relevant portions of the image (e.g., 224a-224b, FIG. 2C).

For each layer, every region and feature may then be assigned a specialized reconstructive super-resolution GAN (layers 314, 316, 318), based on cellular attributes, morphology, coloration, pathology state, etc., from a palette of such specialized GANs (described in more detail below). In this and other ways, the metadata is not merely descriptive, but may also be instructive.

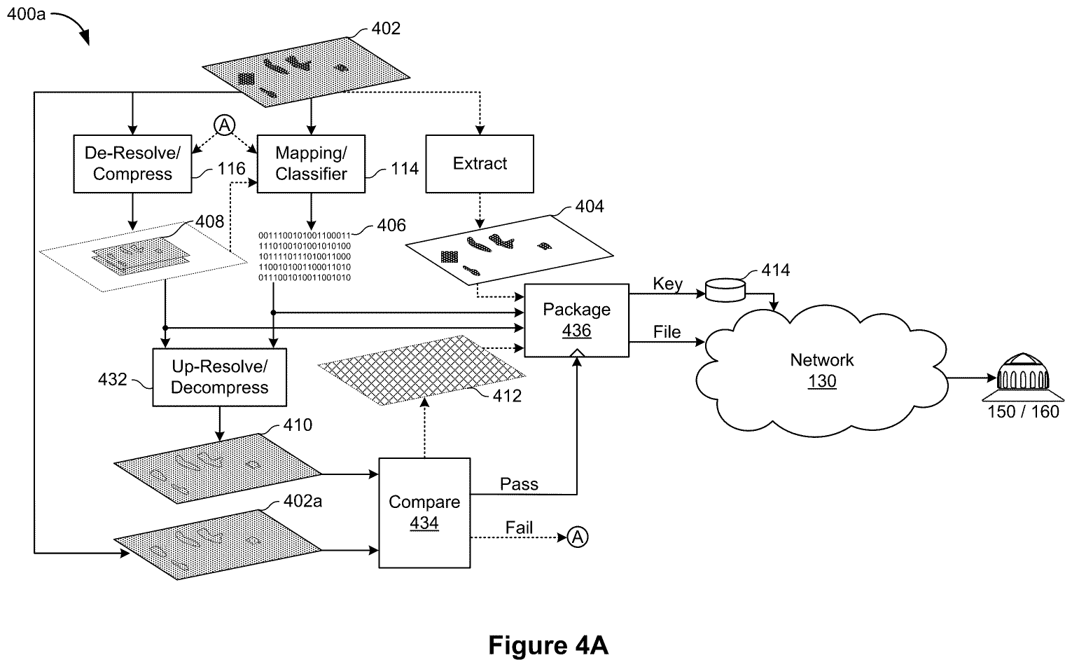

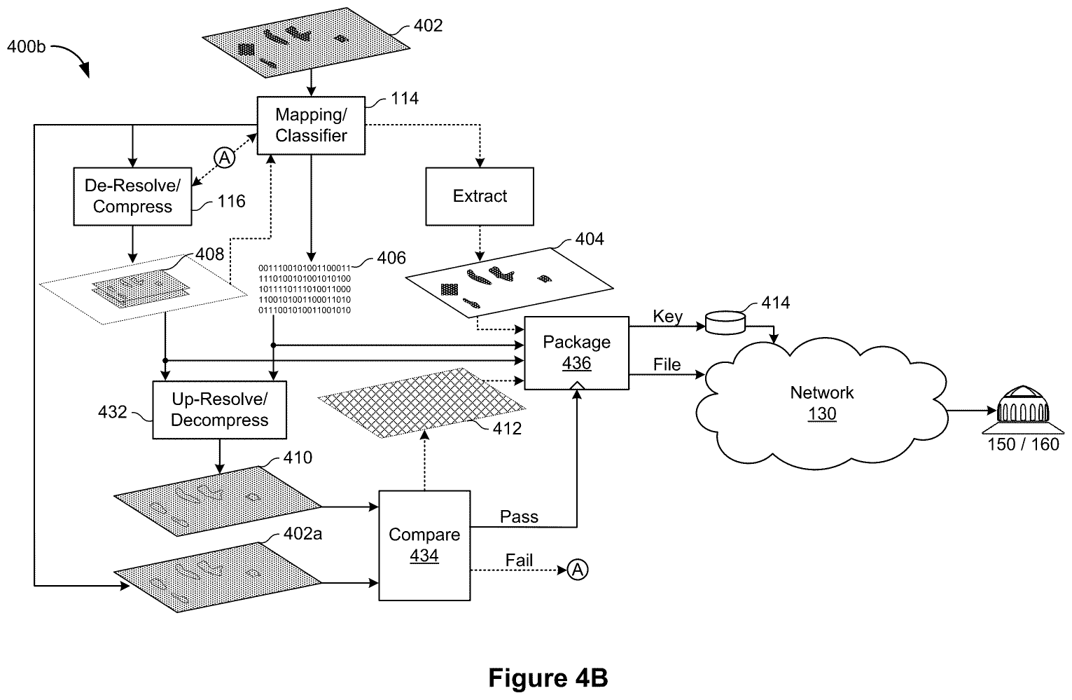

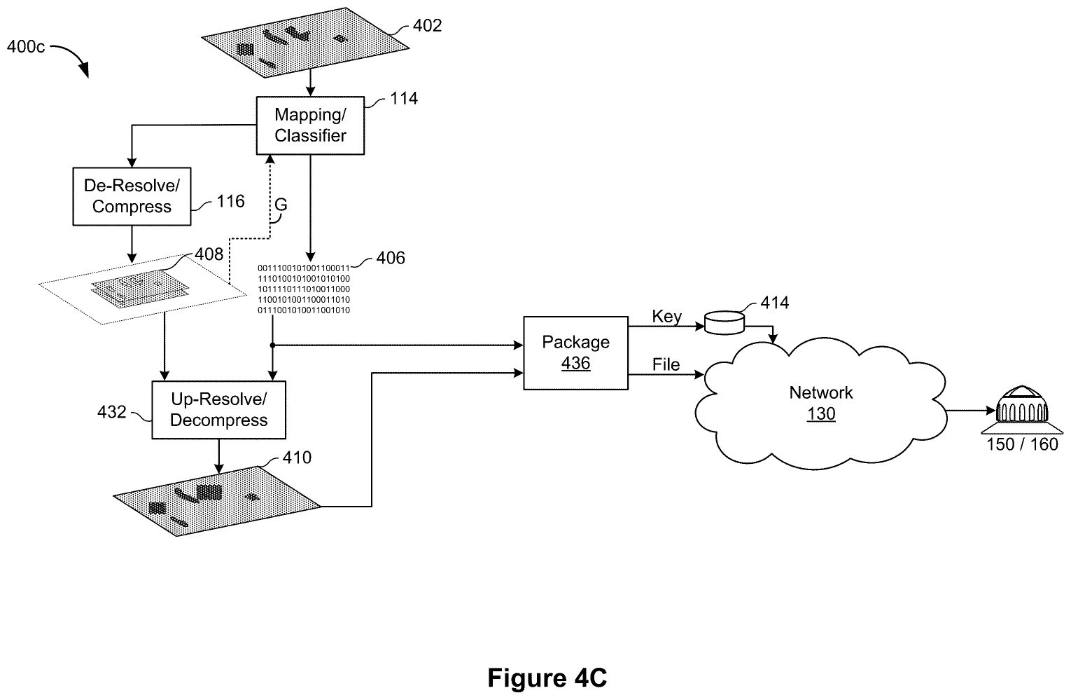

FIG. 4A is a diagram of a de-resolution, compression, and pre-validation process 400a in accordance with some implementations. In some implementations, process 400a is performed by image processing platform 110 of the server system 102, including image mapping/classifier module 114 and image compression module 116.

A source image 402 (e.g., corresponding to image 210, 212, or 214 in FIGS. 2A-2C, or image 302 in FIG. 3) is analyzed by mapping/classifier module 114 to determine regions of diagnostic and/or therapeutic relevance as described above. One or more regions having the highest level of relevance, or levels of relevance meeting a threshold, are optionally extracted into one or more alpha layers 404 (e.g., corresponding to layer 304a in FIG. 3).

Mapping/classifier module 114 generates metadata 406 for each layer. Image compression module 116 de-resolves and/or compresses each of the image layers having a diagnostic and/or therapeutic relevance lower than that of layer 404, or does not meet the relevance threshold, producing one or more down-resolved and/or compressed images 408. Each down-resolved and/or compressed image 408 corresponds to a metadata layer 406, which includes data instructing an up-sampling and/or decompression algorithm how to restore the images. As such, this up-sampling and/or decompression process is pre-validated by up-sampling and/or decompressing (reconstructing) one or more of the down-resolved and/or compressed images 408 using corresponding metadata 406, to generate a reconstructed image 410.

Image processing platform 110 compares the reconstructed image 410 to the original image 402a without the extracted regions 404 and determines a difference between the two images based on the comparison. A difference is to be expected due to the nature of de-resolving, compressing, up-resolving, and/or decompressing an image file (e.g., using lossy algorithms). However, since images 408 were sourced from lower tiers of diagnostic and/or therapeutic relevance, some lost details (e.g., sharpness) may be tolerated due to the accuracy of machine learning/vision models used in reconstructing the image at the client device. These machine learning/vision models are tested at the pre-validation phase, including at this comparison step. If the difference is above a threshold, then the comparison step fails and the process repeats at the de-resolving/compression step.

Upon failing, one or more machine learning/vision models are updated and the image is once again de-resolved and/or compressed using the updated machine learning/vision models. One such example of a machine learning/vision model is a GAN. A GAN uses a generator circuit (e.g., a convolutional neural network (CNN)) to generate images and a discriminator circuit (e.g., another CNN) to determine whether the generated image is real or fake. The reconstruction circuitry and/or algorithm 432 (also referred to as up-resolving and/or decompression circuitry and/or algorithm 432) used for reconstructing image 408 into image 410 may be implemented as a generator network of a GAN, while the comparison circuitry and/or algorithm 434 used for comparing the reconstructed image 410 with the original image 402a may be implemented as a discriminator network of the GAN. As such, each time a comparison results in a failure, the reconstruction circuitry 432 learns and updates its generator model to provide more realistic images 410 in accordance with the updated generator model.

When the comparison between the reconstructed image 410 and the original image 402a passes (e.g., the difference is less than a threshold), then image processing platform 110 packages the extracted layer(s) 404, the de-resolved and/or compressed image(s) 408, and the most recent metadata 406 including the cellular index and the pre-validated, most recent version of the machine vision/learning model (e.g., the GAN model) for use in reconstructing the de-resolved and/or compressed image(s) 408. Images 404, 408, and metadata 406 are packaged into one or more files for transmission over network 130 to one or more client devices 150/160.

In some implementations, the comparison between the reconstructed image 410 and the original image 402a may come close to passing, but not actually pass. Stated another way, the difference may be below the fail threshold, but above the pass threshold by only a threshold amount. Rather than continue to refine the machine learning/vision models and take more time to pre-validate, the difference data 412 itself may be included in the packaged file for transmission over network 130 to one or more client devices 150/160.

In some implementations, each image layer (e.g., 404 and 408) may be de-resolved and/or compressed using different algorithms, depending on which is optimized for the layer. For example, different layers may be compressed using different compression ratios, compression methods, or compression times. Stated another way, each layer may be compressed to a different degree, as well as compressed using a different compression algorithm or type. Since each layer is created based on diagnostic and/or therapeutic relevance, then the re-resolution and compression of the image layers is based on diagnostic and/or therapeutic relevance. Specifically, the more relevant image layers (including the more relevant regions) are de-resolved and/or compressed to a greater degree than the less relevant image layers (including the less relevant regions), and may even possibly use completely different re-resolution and/or compression algorithms.

In some implementations, compression module 116 may create a regional map of gradated variable compression relative to fidelity and diagnostic and/or therapeutic relevance for each image 408. As such, image compression module 116 may perform gradient variable compression relative to diagnostic and/or therapeutic relevance in combination with gradient variable de-resolution relative to diagnostic and/or therapeutic relevance, and subsequently super-resolution of a cellular-indexed medical image using a predefined library of tissue-specific neural networks. These down-resolved and compressed images are a result of several nonredundant complimentary empirical measurements and assessments of the original image.

In some implementations, the use of machine learning/vision models at the reconstruction step 432 involves mapping the indexed features in the cellular index against a library or palette of specialized machine learning/vision models. For example, for implementations using GAN models, the indexed features are mapped against a library or palette of specialized GANs. Stated another way, a GAN or any other specialized tissue-specific, feature-type-specific, or morphology-specific machine learning model may be used to convert parametrically characterized instances of cellular morphology into super-resolved pixels or vector graphic elements which may thereafter become rasterized into pixels. In some implementations, other machine learning/vision models may be used in addition to or as an alternative to GAN models, such as stable diffusion or any other type of machine learning/vision model known or yet to be discovered.

In some implementations, the aforementioned GAN palette can be implemented as any modular library of machine learning/vision super-resolution models, each of which is specialized by cellular and/or tissue-type, state, or morphology. For example, regions 220a-220e in FIG. 2A may be associated with different models in a palette of models, each model specialized to reconstruct whichever cellular and/or tissue type, state, or morphology is present in the respective regions.

The reconstruction process at step 432 in the pre-validation operations in FIG. 4A is repeated at the client device(s) 150/160, using the same metadata and machine learning/vision models; as such, these machine learning/vision models are packaged at step 436 along with the image layers as described above. Thus, a machine learning/vision (e.g., GAN) palette-mapped image may be transmitted to a client device for subsequent decompression and super-resolution.

In some implementations, the pre-validation reconstruction (steps 432-434) implements a final image check to ensure that the image data that is transmitted to the client device(s) may be faithfully reconstructed to the same degree of fidelity as the original image 402. Referring to FIG. 4A, if the check of the reconstructed image 410 is not of sufficient fidelity (“Fail”), then a first loop (following path “A”) isolates discrepant pixels and/or features in the images, tries a different GAN or other machine learning/vision model for the specific discrepant pixels/features, and repeats the check (steps 432-434). This loop may be repeated a number of times until the check passes (“Pass”) and the image file for transmission to the client device(s) is packaged.

In some implementations, after the loop has repeated a threshold of times, or once the difference at comparison step 434 is below a threshold, the original (faithful) pixels that are included in the difference (between images 410 and 40a2) may be isolated into a correction layer 412 which is also packaged into the file for transmission to the client device(s). Additionally or alternatively, a correction GAN may generate a fine-correction alpha layer for the specific layer, region, or entire image that is the subject of the difference (between images 410 and 402a).

In some implementations, the diagnostically and/or therapeutically relevant alpha layer(s) 404, the de-resolved and/or compressed layer(s) 408, the cellular quantification index and other metadata layers 406, a GAN map (or other machine learning/vision map) layer, and an optional correction layer 412 are all packaged within a single file wrapper, isolated, retained, and remotely hosted as a known image check key (KICK) file. In some implementations, the KICK file may amount to approximately 30% of the original file-size, which is a significant improvement for purposes of optimizing finite storage resources by significantly decreasing the storage burden at the server system 102 and at client device(s) 150/160. In some implementations, the original image 402 may be deleted from storage 120 after the KICK file is packaged, being replaced by the KICK file itself and made available for future viewing requests.

In some implementations, the diagnostically and/or therapeutically relevant alpha layer(s) 404, the cellular quantification index and other metadata layers 406, a GAN map (or other machine learning/vision map) layer, and an optional correction layer 412 are packaged as a “Key” file 414, separated from the down-resolved and/or compressed layers 408 of the main packaged file. In some implementations, this key file may be roughly one fourth of the file size of the original image 402, and the main file may also be one fourth of the original image 402, affording the client device(s) a more compelling reduction of their storage burden. Thus, the client device(s) would receive a diagnostically and/or therapeutically relevant key file for super-resolution and a main file for combining with the key file to create a complete image (e.g., looking like original image 402).

The following example illustrates the features described above with reference to FIG. 4A. An AI engine (mapping/classifier 114) performs quantitative mapping of an input slide image 402, cataloguing (classifying) and mapping every feature, morphology, organelle, nucleus, cell orientation, cellular state, and so forth. The most relevant features and/or regions are isolated into an alpha layer 404 and sequestered as pristine elements of the source image (e.g., less than 30% of the total pixels). The remaining features and/or regions are also then isolated into alpha layers, each one being de-resolved and/or compressed based on their respective diagnostic/therapeutic relevance scores. This leverages the fact that healthy tissues are generally more normal and regular, therefore more predictable to a specialized neural network. A final check step validates restoration (comparison 434), adjusting quality metrics until fidelity is perfect (or above a predetermined threshold). Lastly, a reduced-bit-rate correction layer 412 is also created (but only as-needed).

FIG. 4B depicts an alternative implementation of a diagram of a de-resolution, compression, and pre-validation process 400b in accordance with some implementations. Process 400b (FIG. 4B) is identical to process 400a (FIG. 4A), except for the placement and functionality of the mapping/classifier 114.

Process 400b supports a first approach, in which the input image 402 is mapped first by the mapping/classifier 114, which then guides the de-resolution or compression process 116, as well as extraction of the more/most relevant features and/or regions. Specifically, according to the classification and mapping of relevant features and/or regions, module 114 instructs the de-resolving/compression module 116 which features and/or regions to process into corresponding alpha layers, as well as the feature extraction module which portions to extract in the most relevant alpha layer 404.

Process 400b supports a second approach, in which the input image 402 is globally de-resolved at module 116, and the lower resolution images are used for classifier mappings, which then guide the feature extraction and selective variable compression or up/down resolution or other processing (e.g., color reduction). Specifically, based on the globally de-resolved input images 408, the mapping/classifier module 114 can more efficiently determine relevant features and/or regions for extraction and subsequent down/up resolution or compression/decompression, since the de-resolved images 408 have less data to process. This added efficiency saves time and allows input images 402 to be processed more quickly, and at little to no effect on quality.

Process 400b supports any combination of the first and second approaches discussed above, such as a first (simpler) classifier at full resolution (as in the first approach), followed by a more rich/complete classifier at lower resolution (as in the second approach).

FIG. 4C depicts another implementation of a medical image processing scheme 400c in accordance with some implementations. Features of process 400c (FIG. 4C) that are identical to those in process 400a (FIG. 4A) and process 400b (FIG. 4B) are similar labeled.

In process 400c, the input image 402 is divided into a plurality of tiles (unless the tiles are provided from an image scanner). The tile size for each image is based on priorities of speed, quality, and compression potential. Upon being processed by the mapping/classifier 114 (as described above with reference to processes 400a and 400b, the input image (each tile) is de-resolved and/or compressed. Since the entire image portion (e.g., the whole tile) is de-resolved and/or compressed, this step may be referred to as global down-resolving and/or global compression. Thus, the entire image (all of the tiles) is globally down-resolved and/or compressed. In one example, the resulting de-resolved image layer 408 may be 50% or less of the size of the input image 402 (or more in other examples).

In some implementations, a full-resolution image may be reconstituted from portions of the image which have each/all undergone various levels of processing (de-resolving/compression/etc.) by managing them at the tile level. For example, highly processed tiles may be glued together with unchanged tiles. In cases where this would create a noticeable visual artifact, a dithering mask may be used to mitigate the edges of one or more of the modified tiles.

The de-resolved/compressed image data 408 is then up-resolved and/or decompressed back to its original resolution/size, generating an output image 410 having the same resolution and/or size/quality as that of the input image 402. Since the entire image (all of the tiles) is up-resolved and/or decompressed, this step may be referred to as global up-resolving and/or global decompression. As described above with reference to processes 400a and 400b, the up-resolve/decompression module 432 uses a GAN that predictively improves clarity of the image data 408, yielding an output image 410 that is at least as detailed as (and in some cases, more detailed than) the original input image 402.

Concurrent to (in parallel with) up-resolving/decompressing the image data 408 using module 432, the mapping/classifier 114 analyzes (line G in FIG. 4C) the down-resolved/compressed image data 408 to instruct subsequent processing. Specifically, if at least a portion (and in some cases, all) of the image data is subject to feature cataloguing and relevance classification while the image data is down-resolved/compressed, the mapping and classification process is more efficient, thereby saving time in generating a fully mapped and classified output image 410 from an unmapped and unclassified input image 402. In other words, the analysis at mapping/classifier module 114 may be much quicker because it is performed using image data at a lower resolution. Based on the aforementioned analysis of the classifier 114, one or more portions of the input image 402 may be manipulated in order to provide higher quality portions of the input image corresponding to diagnostically/therapeutically relevant features.

While the up-resolved/decompressed output image 410 has the same resolution and quality as the input image 402, the output image 410 is more compressible because run-length encoding works better on the resulting (clarified image). Also, in some implementations, the re-up-resolving is performed by filling in predictable pixels (using the GAN or other AI up-resolving process) where the original pixels were deleted during the de-resolving/compression process. This approach provides sharpening in the output image 410 that can be better than the original input image 402. In other words, the missing pixels as a result of de-resolving and/or compression are backfilled with predicted pixels during the up-resolving/decompression process, thereby increasing the number of predictable pixels by simply over-writing previously deleted pixels with pixels that come from the prediction used by the up-resolve/decompression module 432.

The output image 410 is provided (in some implementations, along with metadata layers 406) to client device(s) 150/160 via packaging 436 and the network 130 (as described above with reference to processes 400a and 400b).

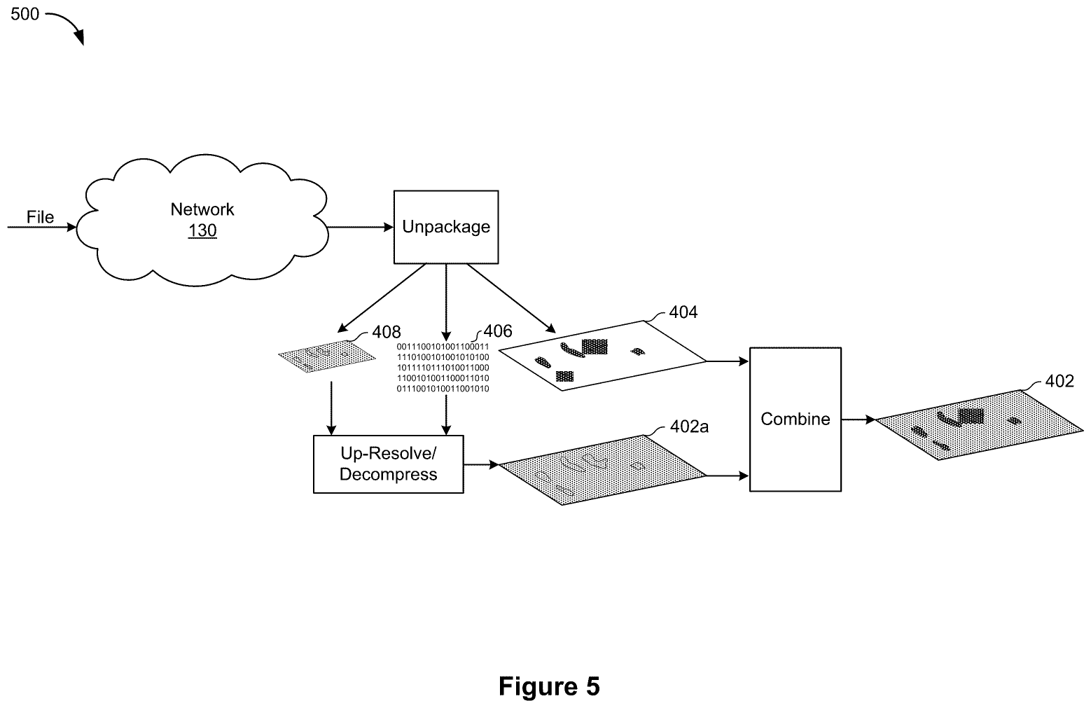

FIG. 5 is a diagram of a super-resolution, decompression, and display process 500 in accordance with some implementations. Process 500 is performed at the client device(s) 150/160 in response to receiving the KICK file or Key and main files from the server system 102 via network 130. The diagnostically and/or therapeutically relevant alpha layer(s) 404, the de-resolved and/or compressed layer(s) 408, the cellular quantification index and other metadata layers 406, GAN map (or other machine learning/vision map) layer, and an optional correction layer are unpackaged for separate processing. The de-resolved and/or compressed layer(s) 408 are super-resolved (also referred to as being up-resolved) and/or decompressed using the GAN map data in connection with metadata 406 (e.g., with the cellular index), thereby producing a reconstructed image 402a (corresponding to the final version of image 402a in FIG. 4 at server system 102 during the pre-validation process), and the reconstructed image 402a is combined with the diagnostically and/or therapeutically relevant alpha layer(s) 404 to produce a restoration of the original image 402, with the same levels of fidelity as the original image acquired by the image acquisition module 112 at server system 102.

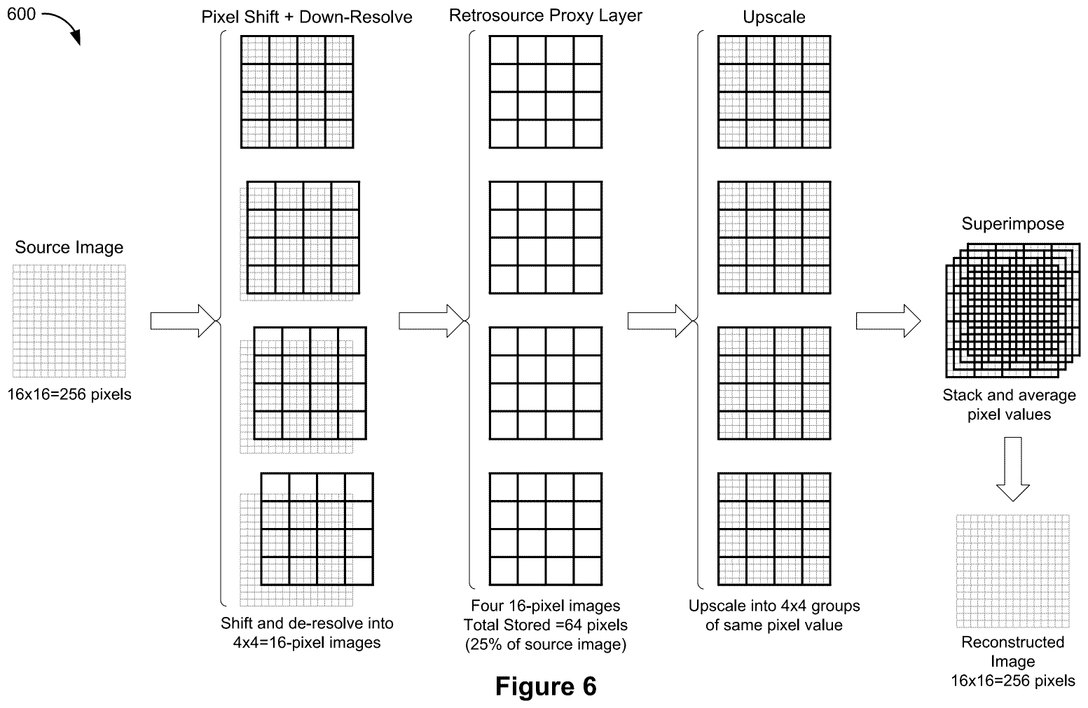

The up-resolving/decompression function uses the cellular index metadata and/or the specialized GAN map (or other machine learning/vision map) received in the file(s) from server system 102 to up-resolve and/or decompress the image(s) 408. As an optional final up-resolution process (e.g., after the combining of images 404 and 402a), the restored image 402 may be further up-resolved beyond the original sensor resolution using the pixel-shifting resolution changing functionality as described herein (e.g., with reference to FIG. 6).

In some implementations, included in the metadata layer 406 is diagnostic workflow instructions, including an order in which to display the relevant regions (e.g., 220a, followed by 220b, followed by 220c, and so forth (FIG. 2A)). In some implementations, the diagnostic workflow is predicted by the TPI or in-house A.I. prediction algorithm(s). As such, not only may the diagnostic outcome be predicted (e.g., in the form of diagnostic and/or therapeutic relevant regions of a medical image), but the workflow for performing the diagnosis (the diagnostic workflow of the pathologist) may also be predicted. Stated another way, the prediction algorithm determines which regions of the medical image the pathologist using client device(s) 150/160 will want to view first, second, and so forth.

By including the diagnostic workflow instructions in the metadata layer 406, image processing platform 110 not only encodes medical images (e.g., whole-slide images), but can also encode a movie or guided viewing of the A.I. predicted workflow of the pathologist, which is usually only 25% to 50% of the total image. In some implementations, the prediction algorithms not only determine the order of regions, but also the level of zoom, the angle, which regions to display adjacent to each other, and so forth.

In some implementations, during the actual diagnostic workflow (while the pathologist is viewing the various regions of the image), additions or revisions to the workflow may be noted at the client device(s) 150/160 and fed back to the predictive model at server system 102. Such additions and revisions may be used to update the predictive workflow models used at server system 102.

In some implementations, the aforementioned additions or revisions may be associated with a user-specific profile, allowing each pathologist to personalize his or her predicted workflow. These user-specific profiles may track an individual relevance model corresponding to the individual pathologist. These user-specific profiles may additionally or alternatively inform the continuous improvement of the global model used at server system 102 for diagnostic workflow predictions for all pathologists. Thus, whereas some systems convert pathology slides into images or even rich data, the disclosed system converts slides into a potent, self-contained diagnostic workflow, which is concise and efficient enough to function across user devices (e.g., smartphones) anytime and anywhere, allowing pathologists to review medical images (e.g., whole-slide images) without being required to travel to an office or use specialized viewing equipment.

For implementations in which an image having original resolution is provided for the client device (e.g., as described above with reference to process 400c in FIG. 4C), up-resolving the received images, unpackaging additional alpha layer(s) 404, and combining them with the up-resolved received images as shown in FIG. 5 is not necessary. In these scenarios, the image itself may simply be decompressed and/or provided directly to the client device.

FIG. 6 is a diagram of a pixel-shifting process 600 in accordance with some implementations. A source image (e.g., 402) is pixel shifted and down-resolved into a plurality of images in a retrosource proxy layer. For example, one group of 256 pixels may be converted to four pixel shifted groups of 16 pixels. For each group of pixels, every pixel is a combined version (e.g., averaged) of the 16 pixels from the source image. The retrosource proxy layer may be the de-resolved image(s) 408 that are packaged and transmitted to the client device(s) 150/160 as described herein with reference to FIGS. 4-5. To reconstruct the image, the retrosource proxy groups of pixels may be upscaled and superimposed (e.g., staked in a pixel-shifted manner) with overlapping pixel values combined (e.g., averaged) to form a reconstructed image.

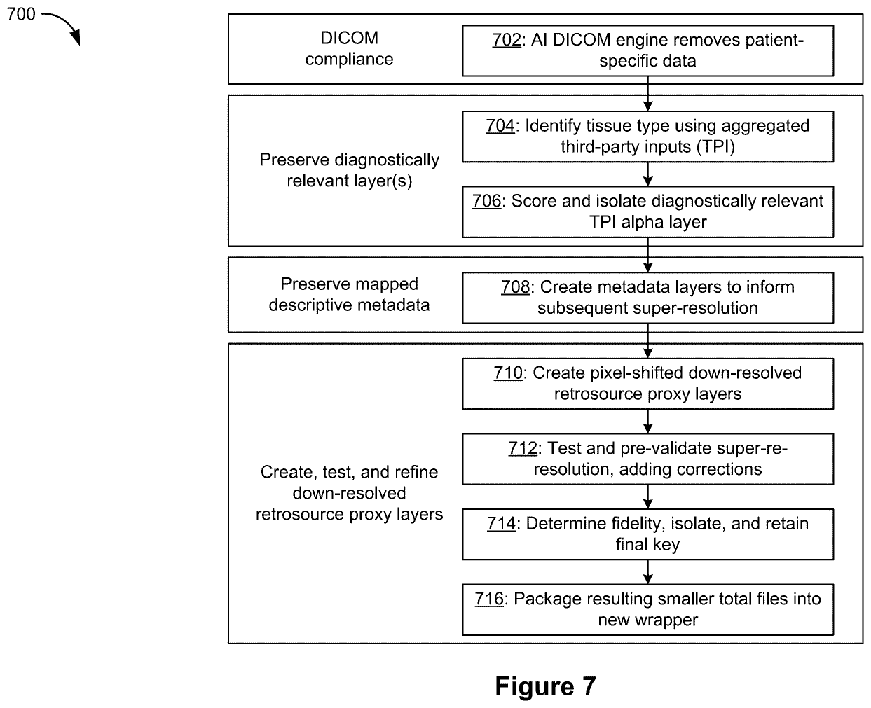

FIG. 7 is a flow diagram illustrating an example process 700 for compressing and transmitting, reconstituting and presenting images for diagnostic annotation in accordance with some implementations. The process may be governed by instructions that are stored in a computer memory or non-transitory computer readable storage medium (e.g., storage 120). The instructions may be included in one or more programs stored in the non-transitory computer readable storage medium. When executed by one or more processors, the instructions cause the server system 102 to perform the process. The non-transitory computer readable storage medium may include one or more solid state storage devices (e.g., Flash memory), magnetic or optical disk storage devices, or other non-volatile memory devices. The instructions may include source code, assembly language code, object code, or any other instruction format that can be interpreted by one or more processors. Some operations in the process may be combined, and the order of some operations may be changed.

Upon obtaining a medical image (e.g., a whole-slide image) (e.g., 402, FIGS. 4A-4C), an A.I. DICOM compliance engine 118 of server system 102 removes patient-specific data from the image. Server system 102 identifies (704) tissue type using aggregated TPI and scores (706) and isolates diagnostically and/or therapeutically relevant TPI alpha layer(s) (e.g., 404, FIGS. 4A-4C). Server system 102 creates (708) metadata layers (e.g., 406, FIGS. 4A-4C) to inform subsequent super-resolution. Server system 102 creates (710) pixel-shifted down-resolved retrosource proxy layers (e.g., 408, FIGS. 4A-4C). Server system 102 tests (712) and pre-validates super-re-resolution, adding corrections (e.g., steps 432, 434 and path “A” in FIGS. 4A-4C). Server system 102 determines (714) fidelity, isolates, and retains a final key (e.g., steps 434 and 436, FIGS. 4A-4C). Server system 102 packages (716) resulting smaller total files into a new wrapper (e.g., step 436, FIGS. 4A-4C).

In some implementations, the input image 402 described above with reference to FIGS. 4A-7 may be part of a z-stack (a plurality of images for each corresponding z height of a specimen). Specimens are typically prepared and imaged by flattening the z-stack into a single layer. By flattening the z-stack, the user loses access to navigation in the z field and any insights that may be observed from the ability to take advantage of such navigation. The following discussion describes implementations for restoration and/or simulating a previously flattened z-stack, providing authentic navigable z field recreation.

In scans where there exists an intact z-stack (plurality of images for each corresponding z height), the image processing platform 110 may capture just the value-adding pixels of a feature relative to the same feature's pixels on the above and beneath layers. Such value-adding pixels may include those in better focus, but the evaluation can also include diagnostic and therapeutic relevance. As such, value-adding pixels may be pixels having a focus corresponding to a predetermined threshold of sharpness, and/or pixels that are part of a feature corresponding to a predetermined threshold of diagnostic or therapeutic relevance.

Since the image mapping module 114 catalogues the entire input image, the image processing platform 110 can determine and save the z-level corresponding to each feature (and portions of each feature) in the image. As a result, the image processing platform 110 can determine which features are behind or on top of other features in the z field. For example, the image processing platform 110 can determine which blood cells are on top of other blood cells, then run one or more predictive GAN models to predict pixel values for obscured portions of the blood cells that are underneath. Thus, the image processing platform 110 can restore a navigable z-field from a flat image.

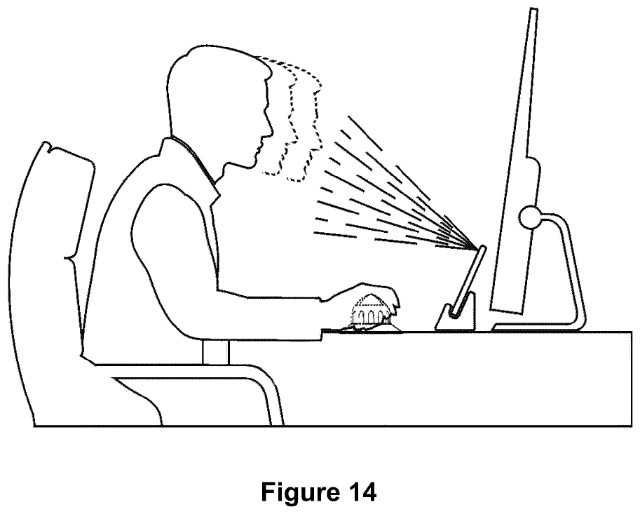

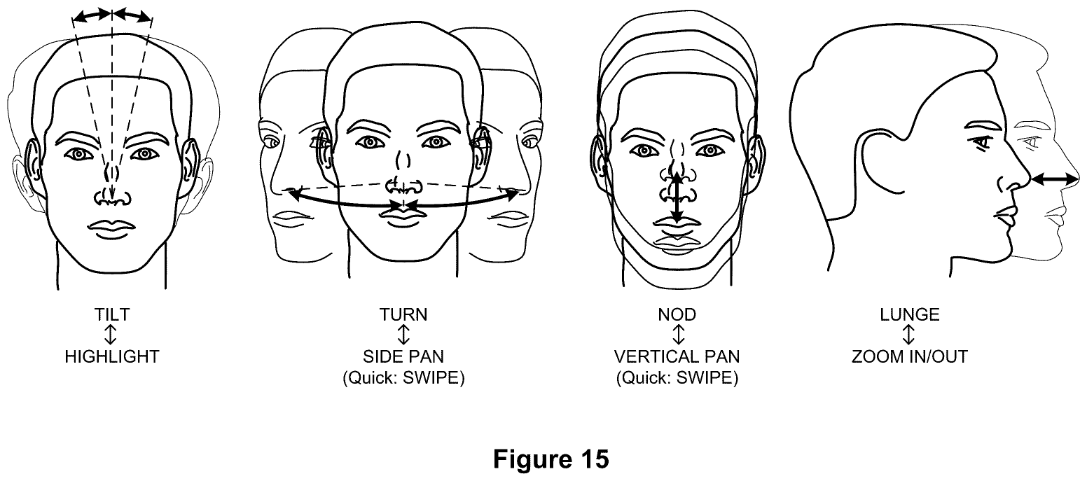

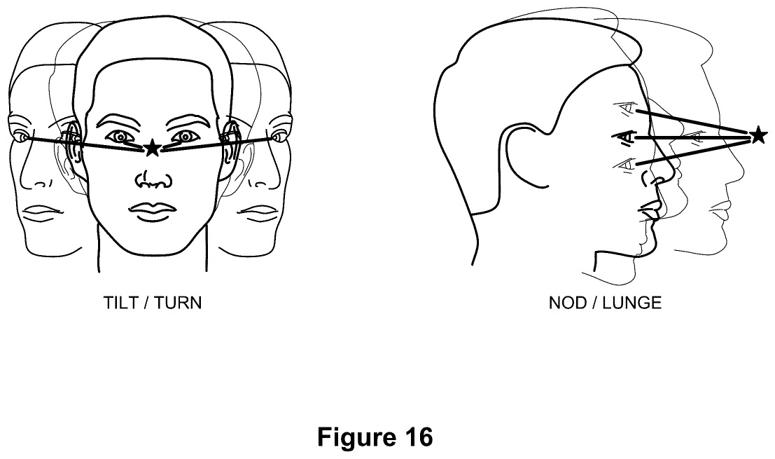

In particular, pixels that are obscured by an overlapping cell or other feature, insofar as those “underneath” pixels differ from the predictive model, may be restored using the techniques described herein. Using this highly economical minority of pixels, retained as an alpha layer, the image processing platform 110 can approximate an authentic navigation experience within the z-axis, either using a control such as a focus knob (e.g., a control on peripheral device 803 described below), or using biometric navigation features (e.g., as described below with reference to FIGS. 14-16). For example, a zoom gesture could give way to z-navigation once it reaches a predefined or dynamically triggered maximum threshold.

In some implementations, using the highly economical minority of pixels described above, retained as an alpha layer, the image processing platform 110 can create a virtual slide at an angle, or even various non-planar virtual surfaces from within the deep-z-field. This feature can be useful for 3D imaging such as lattice light-sheet, or in simulating or superimposing a slide image overlaid on 3D radiology image(s). This use case could include not only features in the slide image well beyond the resolution of the radiology image, but can also include the transferring of stains from the slide image to the adjacent radiology pixels or voxels. From only a few serial sections on slide images, such an approach could virtually generate a 3D “slide image” by interpolating or inferring voxels based on the contextual overlay of the radiology voxels and the slide image pixels. Such a 3D moxel (motion resolution element) could then be utilized as a primary diagnostic workflow exhibit, or could be used as the basis for rendering several “virtual slices” with most or even all of the detail and usefulness that only stained tissue slides could otherwise afford (vs. radiology images).

In some implementations, using the highly economical minority of pixels described above, retained as an alpha layer, the image processing platform 110 can not only determine how deep a plurality of features are in the z-field, but also where they are relative to the focus. Based on that, the image processing platform 110 can calculatably back out other optical aberrations (such as spherical aberrations), can eliminate spectral differences associated with different distances from the focus and different feature geometries, or leave the spectral differences in place to give users an authentic navigation of the z-field. Even further, the image processing platform 110 can put “underneath” features even more behind a given feature by making it even more out of focus, in order to provide even more of an authentic z-navigation effect.

The following section discusses economy of retained pixels by way of predictive models (e.g., GAN mapping). Stated another way, the image processing platform may save only the pixels (in the image data provided to the packager 436) that differ from the various predictions described above with reference to the GAN models in processes 400a, 400b, and 400c (FIGS. 4A-4C).

In some implementations, the image processing platform only retains pixels that differ from the outcomes of the GAN predictions. In some implementations, the image processing platform may selectively replace some pixels in a way that enhances usability. The prediction efficiency can be informed with an ever increasing library of models and parameters to characterize and thereby authentically recreate each pixel/feature.

Specifically, after a predictive GAN-based reconstruction (“restoration”) of the image has been checked for fidelity (e.g., as discussed above with reference to modules 432, 434, and 114) and, in some instances, finely adjusted to maximize fidelity up until it meets a specified threshold for fidelity, the remainder (e.g., final correction layer 412, also referred to as residual coding or error residual coding) can be saved as a distinct file or as a layer in a pyramidal image file (e.g., packaged at step 436), or it could be included with any number of meta-layers contained in the file wrapper.

As the GAN models continue to improve at accurately predicting what the super-resolved representation of any given cell or cellular feature or biomarker looks like, the aforementioned remainder will be smaller and smaller. Thus, the image processing platform may not need to save any pixels except for just the ones that differ from the predictions. This is especially valuable in reducing the file size of a volumetric image (also referred to as a “z-stack”) or the “voxels” of a 3D radiology image. Thus, the image processing platform only needs to save those pixels that differ from the predictions of the generative models . . . which will be ever-increasingly accurate predictions.

This aforementioned “remainder” efficiency is an important part of the image processing techniques described herein, because the largest images at the highest magnifications will conversely have the greatest ratio of pixels (or voxels) per cell. And since the predictive models work best at predictively reconstructing “standard/normal/healthy” cells, that leads to a very high compression ratio.

For example, a typical blood smear is comprised (mostly) of healthy red blood cells and suspect white blood cells. The healthy red blood cells are of very little diagnostic relevance, and yet they may outnumber the white blood cells by 600-to-1. The GAN models as described herein can very accurately predict the more-than 4,500 pixels of each red blood cell based on just 36 concise parameters comprising about 72 bytes of data. This would constitute a compression ratio of about 99.9% for the red blood cell regions of the image. And since the red blood cells outnumber the white blood cells by a factor of 600, that would yield a rough potential compression of 99.9×(600/601). Even for tissue models, the compression ratios could be above 70%.

The aforementioned error-remainder efficiency applies to the volumetric images discussed above, including z-stack slide images and also the resulting volumetric images of a light-sheet microscopy and/or radiology.

Stated another way, any pixels that the various predictive models described herein can accurately predict (models working individually or in any combination) can be de-resolved and faithfully reconstructed thereafter (e.g., as discussed above with reference to modules 116 and 432). This can comprise entire red blood cells, edges of cells or nuclei, or organelles within cells, or chromatids within cells and their respective granularity, Auer rods, mitotic chromosomes, and so forth. This can also comprise having an alpha layer (image region) just for the cells underneath the various-layers within a z-stack (volumetric image). For all of the aforementioned examples, the image processing platform only needs to save those pixels that deviate from the predictive model.

In general, the aforementioned error-remainder efficiency applies to anything that can get imaged, including the readout of an NGS flow-cell, the Karyotype of chromosomes (which sometimes sit atop one-another), the volumetric layers within a lattice light-sheet image, and so forth.

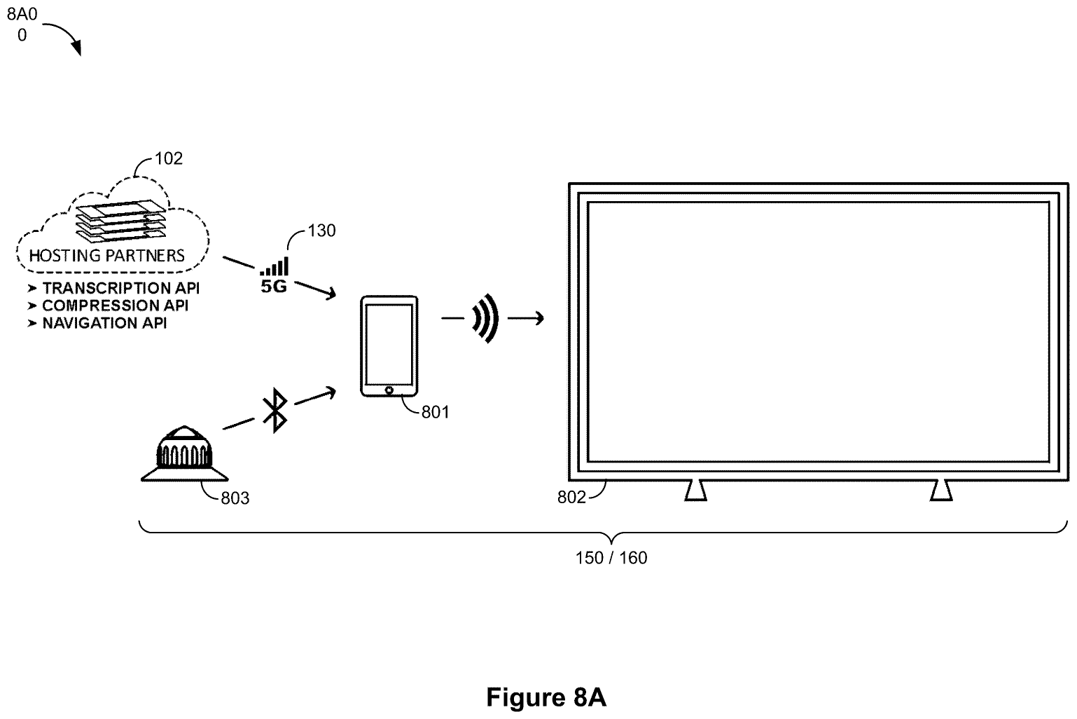

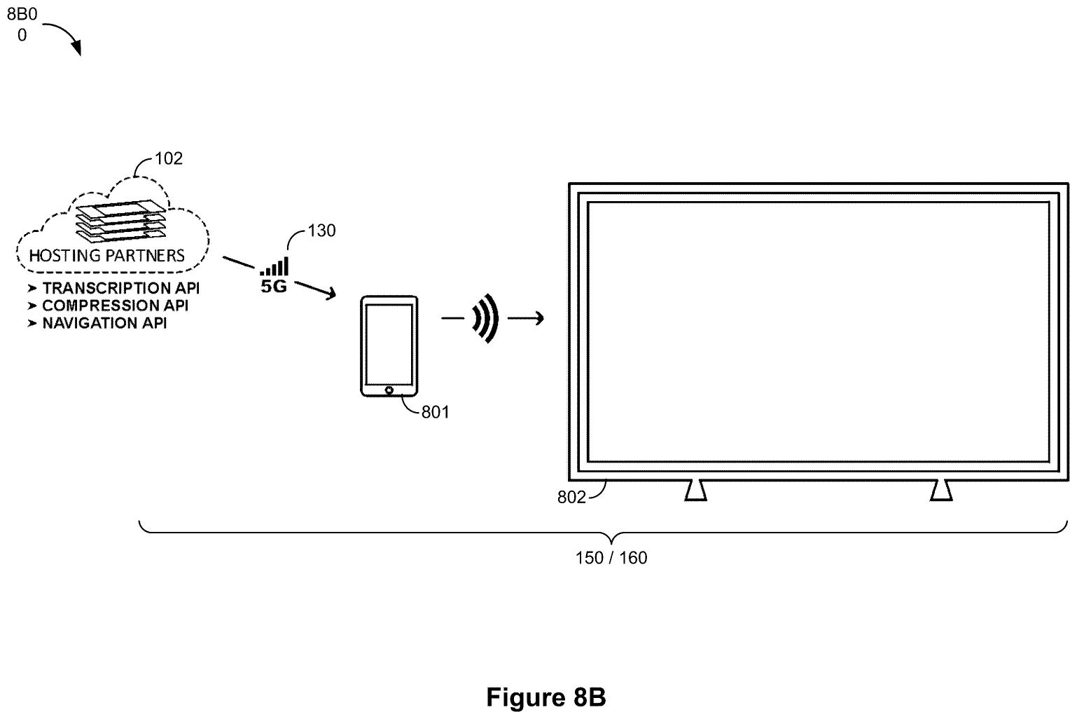

FIG. 8A is a diagram of a system 800 for displaying and interacting with medical images in accordance with some implementations. Server system 102 transmits image files (e.g., 414, FIGS. 4A-4C) to a client device 150/160 via network 130. The client device 150/160 includes, for example, a smartphone 801, and is optionally communicatively coupled to a peripheral device 803 and a display device 802 for interacting with and viewing the restored images (e.g., 402, FIG. 5). In some implementations, such as in FIG. 8B, the peripheral device is unnecessary, and the client device may be the smartphone 801 only, the display device 802 only, or the smartphone 801 coupled to the display device 802.

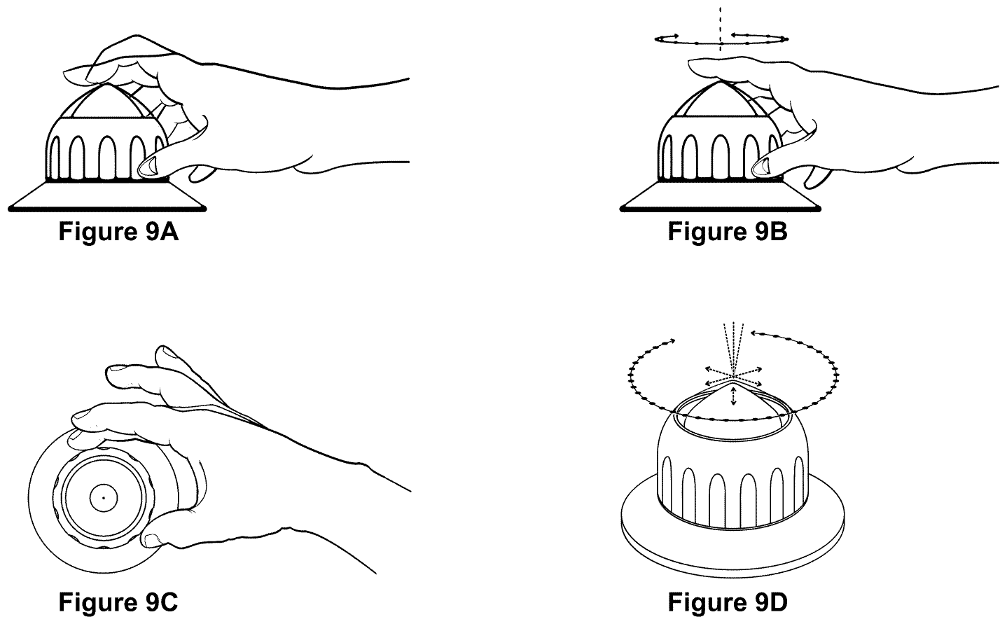

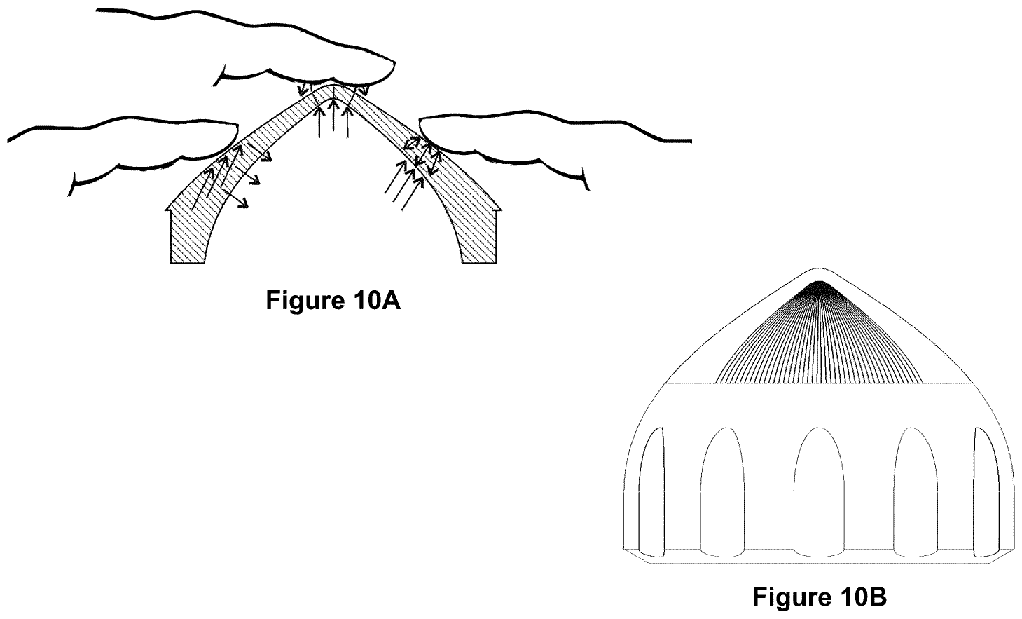

FIGS. 9A-9D and 10A-10B are diagrams of a peripheral device (e.g., 803, FIG. 8) configured for interacting with medical images in accordance with some implementations. The peripheral device depicted in these figures, may be used to advance through image regions as part of a diagnostic workflow specified in the metadata layer (e.g., 406, FIGS. 4A-4C) associated with the image. Additional details regarding the peripheral device are disclosed below.

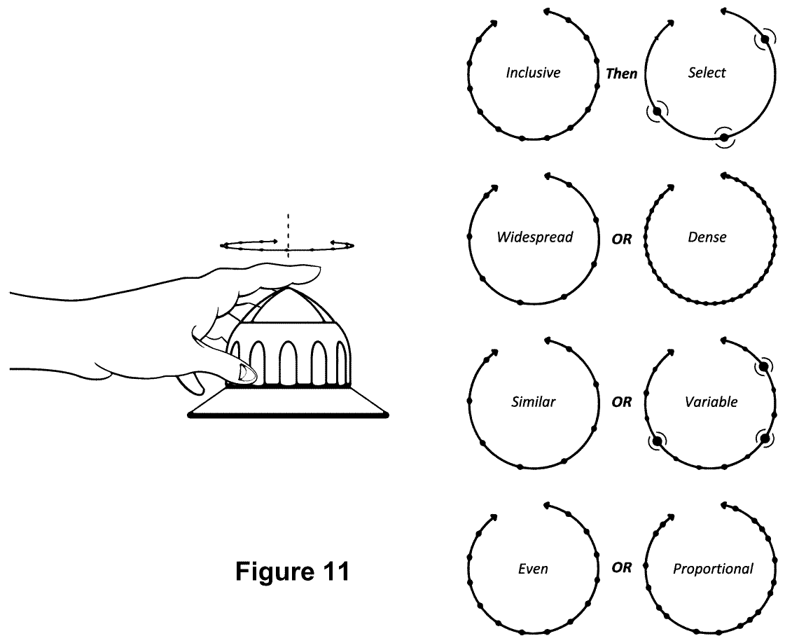

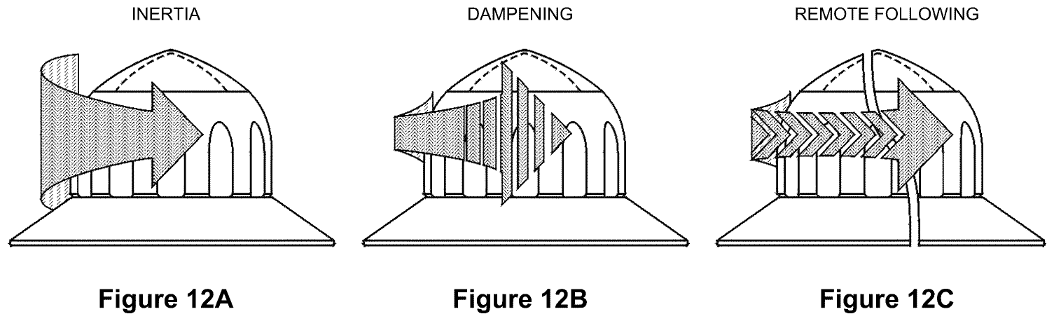

FIGS. 11 and 12A-12C are diagrams depicting a plurality of usage modes of a peripheral device (e.g., 803, FIG. 8) configured for interacting with medical images in accordance with some implementations. Additional details regarding these usage modes are disclosed below.

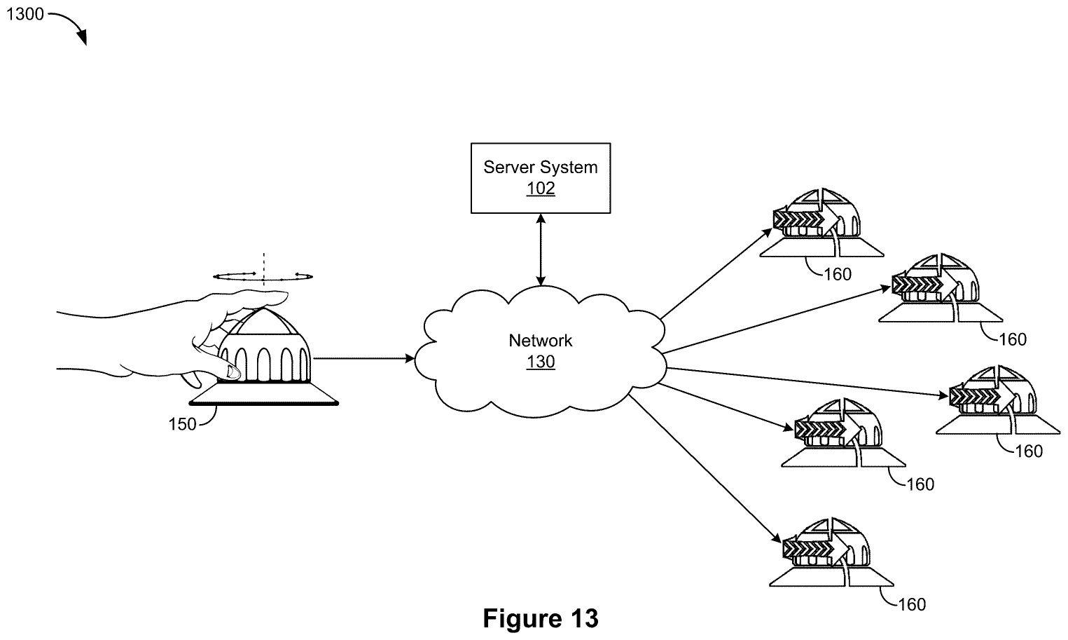

FIG. 13 is a diagram of a system 1300 for collaborative interaction with medical images in accordance with some implementations. In some implementations, movements of a peripheral device at a first client device 150 are transmitted to one or more second client devices 160, thereby causing peripheral devices associated with the one or more second client devices 160 to perform the same movements as the peripheral device at the first client device 150. In such implementations, a lead pathologist (150) may train others (160) to perform diagnostic workflows in a way that allows others to have the same viewing and tactile experience as the lead pathologist as the lead pathologist navigates through the image regions as part of the diagnostic workflow. Additional details regarding these collaborative interactions are disclosed below.

FIGS. 14-16 are diagrams systems for interacting with medical images using facial gestures in accordance with some implementations. In some implementations, facial gestures may the user of the client device may control the viewing and navigation of image regions of the medical image on a display. Additional details regarding these systems are disclosed below.

Some embodiments of the present disclosure improve compression through the use of A.I. to create down-resolved and pixel-shifted pseudo-source or “retro-source proxy layer” images to reconstitute and up-resolve or super-resolve a suitably faithful facsimile of the original high resolution source image or portions of that source such as tiles or regions within tiles or groups of tiles or regions. Such embodiments may also retain reference portions of the original image for a machine learning discrimination circuit in the up-resolution process. Such embodiments may also utilize such reference portions of the original image for a machine learning discrimination circuit in an a-priori verification and/or validation of the up-resolution process.

Some embodiments of the present disclosure improve compression through the use of an A.I. system to analyze the regions and features of a high resolution pathology slide image and/or biopsy specimen to compare it against a continually updated “known tissues library,” said library containing raster and/or vector and/or wavelet data examples of various types of cells, cytoplasm, organelles, vascular formations, tumors, cysts, lumens, glands, lacunae, laminae, and other diagnostically relevant features as described above in various states of existence such as metastasis, mitosis, miosis, carcinogenesis, apoptosis, and so forth. Such a library may contain isolated, specific or general or stochastic parametric data for such examples such as morphology type, area, width, diameter, contrast, rotational state, distortion, aspect ratio, presence of a protein, and so forth. Such a library may contain for each example or parameter of said examples such statistical data as median, mean, standard deviation, and so forth. Such a library may contain for one or more examples therein correlative relationships between the examples an one-another or between the examples and various factors such as are found in patient data and/or metadata. In such an embodiment, the A.I. system records the resulting attributes and parameters relevant to the cells or regions as one or more metadata layers, mapping said metadata against the cartesian coordinates of the regions of the specimen and/or mapping said metadata against the indexed locations of the formations detected in the image or specimen, and/or mapping said metadata against their pixel locations within the image or portions of the image. Such metadata is then subsequently used by the aforementioned A.I. system or by another A.I. system or subsystem in support of the subsequent decompression and/or reconstitution and/or up-resolution or super-resolution of a suitably faithful facsimile of the original image. Such aforementioned metadata may be retained and stored and/or transmitted and/or mined as an image layer, or may be stored steganographically within the pixels of an image layer, or may be retained and stored coincidently as a data file or array such as XML or HTML or as an ASCII text file or DB2 or DBF or CSV or JSON or MDB or other indexable and searchable and/or otherwise mine-able format or data modality.

Some embodiments of the present disclosure improve compression through the use of an A.I. system which concurrently reconstitutes the up-resolved or super-resolved facsimile of the original image using the metadata layers of the previous embodiment's analysis to guide said up-resolution or super-resolution process. Such action serves to confirm that such facsimile is and will subsequently be when re-performed of sufficient fidelity and consistency to and with the original high resolution image. Such an embodiment may employ for such super-resolution a library of distinct and specialized generative adversarial networks (GANs), each specialized to a type of cell, type of tissue, state of tissue or cell, or other useful and distinct and diagnostically relevant aspect of the image and/or specimen or portion thereof. In such an embodiment, the system determines which specialized GAN most accurately approximates the original image or region of said image, and associates or “maps” it to the region or specimen location or feature for which it was verified as effective. Such a “GAN map” is then retained as metadata either indexed as a dataset or retained as an image layer. If retained as an image layer, such metadata may utilize compression such as run length encoding (RLE) as such attributes may likely tend to apply to numerous consecutive or adjacent pixels or regions or specimen features.

Some embodiments of the present disclosure use an A.I. software and/or hardware system to aggregate groups of adjacent pixels from the original image or composite image into pseudo-pixels of lower resolution for a “down-resolved” pseudo-source image (or “retro-source proxy”), and to then repeat the aggregation, shifting the next pseudo-source image by a fraction of one aggregate pseudo-pixel size in an approximation of the traditional “pixel-shift” process. The system verifies that the “retro-source” images faithfully reconstruct the original image when recombined and up-resolved using one or more available algorithms, GANs or other types of neural networks. In such verification, the machine learning system uses the portions of the original source image as the reference in a discriminator circuit or comparative loop to reconstitute a suitably faithful facsimile of the original high resolution source or portions of that source such as tiles or regions within tiles or groups of tiles or regions.

Some embodiments of the present disclosure use an A.I. software system to computationally or algorithmically combine a plurality of optically coincident exposures for the cancellation of sensor noise to create a “de-noised” source image. That de-noised source is then down-resolved by combining adjacent pixels in square clusters of four, nine, or sixteen. This process is repeated to generate a series of down-resolved images, each shifted by a faction of the clustered false-pixels, typically by a shift distance corresponding to one of the original native source pixels. The A.I. system runs the recombinant and up-resolution process, verifying that the product of the newly created pixel-shifted “retro-source proxy” images faithfully recreates or sufficiently approximates the original native resolution and/or the de-noised multi-exposure composite which was the source of the de-resolution and pixels shift process. Such an embodiment affords a file size advantage: The total size of the retro-source proxy layer images are proportional to the number (N) of such images, but the up-resolved result will have a file size proportional to the square of that number (N). The up-resolved result is generated upon demand, whereas the smaller retro-source proxy layer images (individually and in-aggregate) are the version of the subject content which is stored and transmitted. And because the A.I. pre-versifies the fidelity, such embodiments minimize the number (N) of retrosource proxy images needed for sufficient fidelity.

Some embodiments of the present disclosure improve compression through the use of A.I. to select regions of pixels within the tiles of a multi-focus-plane source image(s) a.k.a. a “z-stacked” image set, which represent the preferred image quality and/or diagnostic relevance and/or suitability for a reduced and optimized color palette for any given Cartesian coordinate location of the imaged specimen, aggregating such selected pixels or regions of pixels into a pseudo-source image of specimen features and/or portions which could not have been imaged by the sensor as they did not exist in a coincident plane, or in a coincident line of a line-scanning sensor.

Some embodiments of the present disclosure improve compression through the use of A.I. to select regions of pixels within a source image or within such an aforementioned selectively aggregated pseudo-source image, which represent the preferred suitability for a tissue-specific and/or pathology-specific graphic token palette.

Some embodiments of the present disclosure improve compression through the use of A.I. to select regions of pixels within a source image or within such an aforementioned selectively aggregated pseudo-source image, which represent the preferred suitability for compression by way of run-length encoding.

Some embodiments of the present disclosure improve compression through the use of A.I. to select regions of pixels within a source image or within such an aforementioned selectively aggregated pseudo-source image, which represent the preferred suitability for wavelet compression.

Some embodiments of the present disclosure improve compression through the use of A.I. to select regions of pixels within a source image or within such an aforementioned selectively aggregated pseudo-source image, which, if computationally and/or extracted from that source or pseudo-source would leave a remainder with preferred suitability for one or more types of compression.

Some embodiments of the present disclosure improve compression through the use of A.I. to compare such aforementioned extraction layers with the original source image or partially extracted pseudo-source image or aggregated pseudo-source image and to generate a correction factor which when applied to the extracted layer and/or remainder layer(s) improves the fidelity of the reconstructed and/or up-resolved resultant image.

Some embodiments of the present disclosure improve transmission and cloud-hosted viewing of the stored slide images by selectively caching the more diagnostically relevant image portions or reference tiles in such a location or such infrastructure to afford superior speed or lower latency to the user pathologist during their diagnostic workflow or during a collaborative consultation.

Some embodiments of the present disclosure improve transmission and cloud-hosted viewing of the stored slide images by using an A.I. system or subsystem to predictively pre-load images or portions of images in such a location or using such infrastructure as may directly facilitate one or more suitable collaborative resources.

Some embodiments of the present disclosure improve transmission and cloud-hosted viewing of the stored slide images by selectively preloading a down-resolved whole slide image or portions of that image in such a location or such infrastructure to afford superior speed or lower latency to the user pathologist during their diagnostic workflow or during a collaborative consultation.

Some embodiments of the present disclosure improve diagnostic workflow using an A.I. system or subsystem to select and/or isolate and/or extract and/or preserve a diagnostically relevant reference portion of the original source image or pseudo-source image a-priori. Such portions and the specimen features contained therein are pre-indexed relative to the reconstructed image and those indexed regions are mapped to the detent features of a rotary scroll wheel (e.g., 803) for swift and precise navigation of a great plurality of such features and locations.

Some embodiments of the present disclosure improve diagnostic workflow using an A.I. system or subsystem to afford hands-free navigation, region selection and annotation by way of vocal commands, speech-to-text annotation function, and through eye and face tracking, particularly through the measurement and precise tracking of the vestibulo ocular reflex. The complete navigation and annotation actions may be shared simultaneously with collaborating colleagues or virtual colleagues through the network and across great distance, enabling a real-time consultative services exchange in which diagnostic services may be aggregated and conveyed to those people and regions where such resources are in short supply. Such real-time collaborative diagnosis is distinct from a second opinion network such as Soenksen (U.S. Pat. No. 11,211,170) in that it affords increased skills growth and credibility for the more junior and/or non-western personnel. Such mentorship is vitally important in raising the quality of care, both actual and perceived, within emerging nations and/or economically challenged communities.

Some embodiments of the present disclosure improve the visual quality and reconstructive fidelity of the resulting image using an A.I. system or subsystem preferentially suited and/or dedicated to a specific type or combination of types of tissues, morphologies and/or pathologies. One embodiment of such a specialized system may include a generative adversarial network (GAN) for the up-resolution of cells of a given tissue type which have been determined to exemplify pleomorphism. Another embodiment of such a specialized system may include a generative adversarial network (GAN) for the up-resolution of healthy and regular cells of a given tissue type. Another embodiment of such a specialized system may include a generative adversarial network (GAN) for the up-resolution of cells of a given tissue type which have been determined to exemplify metastasis.

Some embodiments of the present disclosure improve the visual quality and reconstructive fidelity of the resulting image using an A.I. system or subsystem to map pixels or groups of pixels or portions of an image or pseudo-image or cells or groups of cells or Cartesian coordinates or defined regions of coordinates of the imaged specimen against a library and/or palette of such aforementioned specialized tissue-specific GANs and/or morphology-specific GANs or otherwise specialized GANs.

Some embodiments of the present disclosure improve the visual quality and reconstructive fidelity of the resulting image using an A.I. system or subsystem to map pixels or groups of pixels or portions of an image or pseudo-image or cells or groups of cells or Cartesian coordinates or defined regions of coordinates of the imaged specimen against a library and/or palette of tissue-specific and/or morphology-specific or otherwise specialized graphic tokens. In one such embodiment, said specialized token library may be dynamically updated using an A.I. system or subsystem which detects repeated and/or widespread incidence of a potential new graphic token and uses a GAN to establish a new such token, appending it to the extant token library and/or token palette. Such a system may then retroactively apply the improved and/or expanded token library to previously processed images or portions of images, using a discriminator network and reference portions of said images to ensure superior and/or satisfactory resulting reconstructed image quality.

Some embodiments of the present disclosure improve compression through the use of A.I. to select regions of pixels within a source image or within a selectively aggregated pseudo-source image, which, if computationally extracted from that source or pseudo-source would leave a remainder with preferred suitability for reduced depth of color including but not limited to optimally palletized color for Hematoxylin and Eosin stain (H&E) or other stain.

Some embodiments of the present disclosure improve compression through the use of A.I. to select regions of pixels within a source image or within a selectively aggregated pseudo-source image, which, if computationally extracted from that source or pseudo-source would have preferred suitability for reduced depth of color including but not limited to optimally palletized color or algorithmically compressed color profile for tissues treated with Hematoxylin and Eosin stain (H&E) or other stains.

Some embodiments of the present disclosure improve diagnostic workflow using a camera and an A.I. system or subsystem to afford hands-free navigation, region selection and annotation by way of vestibulo ocular reflex in which the user's gaze is deliberately fixed upon a selector element such as a cross-hair or selection box or color-highlighted area, line, circle, point or polygon or dimmed or flickering or scintillating-highlighted area, line, circle, point or polygon, and said user's head and/or face is then deliberately moved so as to indicate the intent to shift the image or portion of the image into and/or beneath the aforementioned selector element. The aforementioned camera and A.I. system detect such VOR activity and shift the displayed image accordingly. In some implementations, tracking of the user's head and/or face may be disengaged or disabled in response to receiving a user input temporarily or permanently disabling the head and/or face navigation features. The user can, during this time, re-center his or her face (re-establish a new origin) and re-enable head and/or face tracking via a second user input instructing the system to resume head and/or face tracking.

Some embodiments of the present disclosure improve diagnostic workflow using a camera and an A.I. system or subsystem to afford hands-free highlighting and annotation by way of vestibulo ocular reflex in which the user's gaze is deliberately fixed upon a portion of the displayed image, and said user's head and/or face is then deliberately moved so as to indicate the intent to highlight or select that portion of the image. The aforementioned camera and A.I. system detect such VOR activity and select or trace or select that portion of the displayed image accordingly. Subsequent voice-to-text capture annotates the actively selected region or specimen feature as the pathologist indicates verbally.

Some embodiments of the present disclosure improve diagnostic workflow using a camera, a microphone and an A.I. system or subsystem to afford hands-free navigation, highlighting and annotation by way of vestibulo ocular reflex (VOR) in conjunction with vocal commands such as “highlight,” “select,” “deselect,” “annotate,” “encircle,” “ensquare,” “navigate,” “polygon,” “spline,” “touch-paint,” “new layer,” “mark,” “pin here,” “pause,” “save spot,” “compare-with,” “split-view” or other such commands typical of graphic editing and/or text editing.

Some embodiments of the present disclosure improve diagnostic workflow using a system or subsystem comprised of a camera, a microphone and an A.I. software system to afford hands-free navigation by way of head and/or facial movements in conjunction with a vocal command such as “vanity-mirror.” In such a mode, the user indicates intent to increase image magnification by leaning-in toward the display. The system detects and tracks this movement in real-time, adjusting the displayed image accordingly. Similarly, the user indicates intent to pan left by turning their head to the left, or indicates pan up by tilting their head up, down by tilting their head down. The system detects and tracks this movement in real-time, adjusting the displayed image accordingly. In such a “vanity-mirror” mode, the user may verbally instruct the system to apply or increase or decrease a “scaling-factor” so that a gentle movement may induce a great shifting of the displayed image, or vice-versa. Similarly, the user may, in the aforementioned mode, verbally instruct to “reverse” the relationship between their head movements and the resulting shifts of the displayed image. In such a “vanity-mirror” mode, the user may verbally instruct the system to apply or increase or decrease a “stabilization-factor” so that the displayed image is shifted in a smooth and jitter-free fashion without regard to the more minute and/or less seemingly deliberate motions of their face or head. Such a stabilization would be an important feature for user with a degenerative neuro-muscular condition. In some implementations, tracking of the user's head and/or face is disengaged in response to receiving a user input temporarily or permanently disabling the head and/or face navigation features. The user can, during this time, re-center his or her face (re-establish a new origin) and re-enable head and/or face tracking via a second user input instructing the system to resume head and/or face tracking.

In some implementations, the user's eyes' gaze is fixed upon a fixed element being displayed, and the concurrent motion of the face and head is used to instruct the movement of a moved element being displayed and/or the selection of a selected element, which may also be associated with an actuation command.

In some implementations, the instructed movement is of an image relative to a fixed cursor, selection box, painting tool, mask-designator, magnifying selection zone or area-designating graphic display element.

In some implementations, the instructed movement is of a cursor, selection box, painting tool, mask-designator, magnifying selection zone or area-designating graphic display element relative to a fixed image or portion of an image or specimen region.

In some implementations, the instructed movement is of a filename, folder name or an icon or thumbnail representing a file or folder or plurality thereof, relative to a fixed cursor or selection box or magnifying selection zone or file-designating or folder-designating graphic display element.

In some implementations, the instructed movement is of a cursor or selection box or magnifying selection zone or file-designating or folder-designating graphic display element relative to a fixed filename or folder name or icon or thumbnail representing a file or folder or plurality thereof.

In some implementations, the instructed movement is of a command or command list or hierarchical command category or icon or thumbnail or preview representing a command or command category or plurality thereof, relative to a fixed cursor or selection box or magnifying selection zone or file-designating or folder-designating graphic display element.

In some implementations, the instructed movement is of a cursor or selection box or magnifying selection zone or file-designating or folder-designating graphic display element relative to a fixed command or command list or hierarchical command category or icon or thumbnail or preview representing a command or command category or plurality thereof.

In some implementations, the instructed movement is of a setting or settings list or hierarchical settings category or icon or thumbnail or preview representing a setting or settings category or plurality thereof, relative to a fixed cursor or selection box or magnifying selection zone or setting-value-designating or setting-selecting graphic display element.

In some implementations, the instructed movement is of a cursor or selection box or magnifying selection zone or selection-designating or setting-designating graphic display element relative to a fixed setting or settings list or hierarchical settings category or icon or thumbnail or preview representing a setting value or settings category or plurality thereof.

A version or (each/all of the preceding claims) in which the actuation command is voice-command activation, a push-button, scroll wheel, keystroke, touch-pad, touchscreen, deliberate eye-blink, foot-switch, roller-ball, non-verbal sound activation

Some embodiments of the present disclosure improve diagnostic workflow using an A.I. system or subsystem comprised of a smart phone in conjunction with a two part phone cradle, the lower base remaining stationary, and the movable upper cradle holding the phone horizontally with the display facing upward. As the user manipulates the phone with their fingertips, the system displays the magnified slide image on the phone's display as if the phone were an extreme magnifier being slid around upon the actual specimen, or conversely as if a slide were being slid around beneath an optical microscope. The rear camera of the smart-phone senses the movement of the lower base passing beneath it, which may be illuminated as needed by the rear LED of the smart-phone. Commutation may be additionally sensed using the onboard sensors of the phone, or by a Bluetooth paired peripheral with similar functionality, features and construction as a wireless optical scroll-mouse. The upper cradle may facilitate smooth and precise movements by way of low friction pads and/or rollers between the upper cradle and the lower base.

Some embodiments of the present disclosure may implement the aforementioned “table-top” mode in which the lower base is the surface of a table or desk, and the aforementioned scroll-mouse features are integral to the upper cradle.

Some embodiments of the present disclosure may implement the aforementioned “table-top” mode in which the upper cradle is a typical smart-phone case.

Some embodiments of the present disclosure may implement the aforementioned “table-top” mode in which the smart-phone is configured in a tilted state or adjustably tilted state, i.e. not parallel to the underlying surface. Such a tilt may be oriented to facilitate more effective face tracking by the front-facing camera of said phone.

Some embodiments of the present disclosure may implement A.I. diagnostic recommendations as draft annotations which the user may affirm, revise or reject at their discretion. Such draft pre-annotations may be presented in synonymous fashion to the annotations of a collaborating colleague. Such annotations may be presented with no distinction between the recommendations of one or more human colleagues, or the recommendations of an A.I. “virtual pathologist,” or the anonymized previous annotations of that same user/pathologist. The system may re-submit to the user a previously evaluated slide so as to authentically measure self-concordance. Such a concordance test may be conducted by the system surreptitiously for some portion of the workflow process.

Some embodiments of the present disclosure may implement the aforementioned “table-top” mode in which the progressive selection, review and annotation of the pre-identified diagnostically relevant specimen features is controlled by facial movements and/or VOR and/or the touch screen of the phone. In such a modality, the user may conceivably complete the entire diagnostic workflow without taking their hands from their grasping hold upon the phone as proxy for a traditional slide manipulation.

Some embodiments of the present disclosure may implement the aforementioned facial tracking navigation in manner which smoothly progresses through the various pre-identified diagnostically relevant features, cells, locations or annotations.

Some embodiments of the present disclosure may implement the aforementioned facial tracking navigation in manner which progresses through the various pre-identified diagnostically relevant features, cells, locations or annotations in a non-linear fashion, “snapping” or “Popping” to each indexed feature or location or annotation in a similar fashion to the navigation behaviors associated with the aforementioned scroll wheel embodiment. Such “snap” or “pop” navigation would serve to expedite the user's review of the specimen and image. In such navigation, audible and visual cues would denote the progressive selection of the respective locations or features, such cues determined contextually by the system and/or by user configurable settings. In such embodiments, the system may temporarily or persistently alter the sensitivity and/or scale of the facial movement tracking to facilitate a more stable or fluent review experience for the user. In such embodiments, the navigation may proceed between features and/or locations while remaining at or about a single magnification, or may alternately reduce magnification before proceeding to the next location, or alternately the navigation may proceed so as to visually approximate an apparent flying or bounding arc in the z axis. In such an embodiment, the system may descale, or attenuate or disregard entirely the forward lunge aspect of the facial tracking in a manner that is momentary, or temporary, or persistent or modal.

Some embodiments of the present disclosure may implement the aforementioned “table-top” mode while Miracasting the display output to a television.

Some embodiments of the present disclosure improve diagnostic workflow using a system or subsystem comprised of a camera, a microphone and an A.I. software system to afford hand-tracking or hands-tracking control of navigation, highlighting and annotation.

Some embodiments of the present disclosure improve diagnostic workflow using a system or subsystem comprised of a touch-sensitive sensor, a camera, a microphone and an A.I. software system to afford the aforementioned control modalities in any combination in conjunction with a touchscreen such as smart-phone laid upon a desk or table-top surface or held in a vertical or semi-vertical cradle.

Some embodiments of the present disclosure improve diagnostic workflow using a system comprised of an A.I. software system or subsystem, and a 5G smart-phone wirelessly interfaced with a nearby large-screen television in a display mode known as “Miracasting”. In said embodiment, the slide-image is cloud-hosted and streamed via 5G mobile network. Voice-commands and Voice-to-text transcription of annotations is accomplished by function of the smart-phone. VOR and Face-Tracking navigation and/or selection is also accomplished by way of the smart-phones one or more cameras and/or infra-red tracking dot pattern projector, and accordingly displayed on the television.

Some embodiments of the present disclosure improve diagnostic workflow using a system comprised of an A.I. software system or subsystem, and a 5G smart-phone wirelessly interfaced with a nearby large-screen television, and one or more Bluetooth or WiFi peripheral devices paired with the smart-phone, such as a scroll-wheel, a joystick, a foot-switch or variable foot-pedal, a trackball, a simple selector button, a mouse, a keyboard, a capacitive proximity sensor, an infra-red or ultrasonic motion detector or proximity sensor, one or more discrete or integrated motion-sensing MEM's, accelerometers or strain-gauges, a stylus, a mouse, a haptic VR glove, a wand, a laser pointer, VR/AR display goggles, one or more speakers, one or more LED or LCD displays, headset microphone and/or headphones, remote-control handset, a reflective or fluorescent ball or tape or other sensory or motion-capture control or feedback device such as are commercially available for mobile or desktop computing.

Some embodiments of the present disclosure improve diagnostic workflow using a system comprised of an A.I. software system or subsystem, and a 5G smart-phone wirelessly interfaced with a nearby large-screen television, and a detented scroll-wheel. In such an embodiment, each detent is indexed to a specific feature or region of diagnostic relevance within the imaged specimen, such as a tissue feature, and/or Cartesian coordinate within the imaged specimen, and/or highlighted or annotated portion of the image, and/or annotation or external message or hyperlinked external document or portion of a document or media file or active chat session or collaborating resource. Such a scroll-wheel modality affords uniquely precise yet swift and efficient navigation of large numbers of discrete regions or features or image portions.

Some embodiments of the present disclosure improve diagnostic workflow using a dynamically detented scroll-wheel, said device altering the audio-visual and haptic behaviors of each indexed feature or region as they are reviewed and annotated, in such a manner as to indicate progress and afford review and/or revision of that progress. Such a dynamic scroll-wheel, comprised of a brushless DC motor integrated with one or more electronic circuit boards and a rotating knob or wheel, may also include LED's, push-buttons, membrane buttons, touch sensors, OLED or LCD displays, a palm rest, a speaker or sound transducer, hall-effect sensors or strain gauges, microphones or piezo-electric transducers or sensors, optical or magnetic commutation sensor or other elements of peripheral devices commercially available for mobile or desktop computing.

Some embodiments of the present disclosure improve diagnostic workflow using a dynamically detented scroll-wheel, which uses a brushless DC motor for simulated and dynamically configurable kinetic behaviors such as momentum, resistive inertia, and soft dampening. Such an embodiment may incorporate strain gauges or other sensors in the base of the scroll-wheel to detect manual force axially applied to the top center of the knob for the intent of XY navigation. Such sensors may also detect a tapping action upon the top of the scroll wheel for select and deselect functionality.

Some embodiments of the present disclosure improve diagnostic workflow using a dynamically kinetic scroll-wheel, said device dynamically altering the apparent inertia and/or soft-dampening of the wheel or knob by way of a brushless or brushed DC motor or stepper motor or actuated mechanical feature such as a friction element in conjunction with a servomotor, solenoid or electro-magnet or electronically actuated ferromagnetic fluid. In such an embodiment, the scroll-wheel may move under the control of a remote collaborating colleague or so as to indicate their progress for collaborative diagnostic and/or training purposes. Such aforementioned simulated inertia may afford a customization of the feel of the device for better suitability to a variety of users or to reduce hand and wrist fatigue. Said embodiment may also afford strength of detents by user-setting for similar reasons of fatigue and/or user preference.

Some embodiments of the present disclosure improve collaborative diagnostic workflow using an A.I. system or subsystem to select and interconnect one or more collaborate and available resources from a real-time registry of currently active pathologists and/or virtual pathologists within a cloud-hosted network. In such an embodiment, the aforementioned dynamic scroll-wheel may be used to garner simultaneous feedback of an opinion or rating from a plurality of pathologists and/or virtual pathologists, which is tabulated and/or aggregated by the system whether computationally or by neural network, according to one or more concordance algorithms or other preferred standards and practices. In such a mode, the scroll-wheel may serve as a remote tactile and haptic hand-shake by and between collaborating participants. Such collaborative sessions may be recorded for subsequent review.

Some embodiments of the present disclosure improve self-concordance using an A.I. system or subsystem to measure and evaluate the differences between the pathologist's diagnostic preferences and tendencies in the diagnosis of similar or same specimen images. Such re-review of previously diagnosed slide images may be induced by the system in the context of a training consultation. Cases of specimen similarity and concordance may be based upon inter-specimen similarities in the system's own classification of their diagnostically relevant attributes, or according to the standards and practices of a medical board or other governing regulatory body. The system may facilitate improvements in self-concordance or facilitate consistent diligence of the pathologist's review by way of subtle suggestive scintillation of the nearby image portions of diagnostic relevance. The system may continually refine its own criteria for diagnostic relevance to more closely approximate the judgement and workflow patterns of the individual pathologist, until nearly every slide review is an exercise in affirmation of the recommended annotations and diagnostic conclusions. This value proposition is a productivity boost, rather than a “replacement” of the pathologist.

Some embodiments of the present disclosure improve self-concordance using an A.I. system which compares the diagnostically relevant features of each slide under diagnosis with previous similar features, images, and cases diagnosed by that pathologists and/or other well-regarded pathologists and/or board standards and practices. Such an embodiment may pre-populate annotations with recommended text, which the user is free to affirm, revise or reject. Such a system, through continual monitoring of actual diagnostic workflow and machine learning, may come to eventually approximate human diagnostic acumen to a sufficient degree for indistinguishable parity in terms of sensitivity and specificity.

The following description includes a plurality of implementations of systems and methods for implementing the concepts described above with reference to FIGS. 1-16. These implementations are provided for as nonlimiting examples.

In some implementations, a method comprises, at a server system, obtaining an image of a specimen (e.g., includes composite images derived from a volumetric z-stack, comprised of pixels, regions, or features selected for diagnostic or therapeutic relevance) (e.g., does not necessarily require a slide; tissue ribbon may be directly scanned without being mounted to a slide) (e.g., including specific circumstances associated with the protocols of one or more pharmaceutical trials and the matching of suitable participant candidates thereto); identifying one or more cellular morphologies of the specimen; mapping a plurality of regions of the image corresponding to the one or more cellular morphologies; assigning a level of diagnostic or therapeutic relevance to each region of the plurality of regions; compressing the plurality of regions using, for each region, a level of compression inversely related to the assigned level of diagnostic or therapeutic relevance for the region (e.g., or a type/method of compression, inversely related with regard to fidelity); receiving a request to view the image from a first client device; and in response to receiving the request to view the image from the first client device, transmitting (i) the compressed plurality of regions and (ii) metadata including an index of the assigned levels of diagnostic or therapeutic relevance of the plurality of regions to the first client device.

In some implementations, assigning the level of diagnostic or therapeutic relevance includes: submitting the image to one or more diagnostic machine vision systems (or human pathologist review); in response to submitting the image, receiving diagnostic or therapeutic relevance data associated with the plurality of regions from the one or more diagnostic machine vision systems; and aggregating the diagnostic or therapeutic relevance data received from the one or more diagnostic machine vision systems; wherein the assigning of the level of diagnostic or therapeutic relevance is based on the aggregated diagnostic or therapeutic relevance data received from the one or more diagnostic machine vision systems (there may be various methods for combining several such inputs for best sensitivity, specificity, and concordance).

In some implementations, the method further includes extracting the plurality of regions into a plurality of discrete alpha layers or images, wherein compressing the plurality of regions includes compressing the plurality of discrete alpha layers or images; associating portions of the metadata with each of the plurality of discrete alpha layers or images; and respectively encoding or encrypting the portions of the metadata into the plurality of discrete alpha layers or images.

In some implementations, identifying the one or more cellular morphologies of the specimen includes compiling a cellular index of features of the image using a predefined library of tissue-specific or pathology-specific neural networks.

In some implementations, assigning the level of diagnostic or therapeutic relevance to each region includes assigning a plurality of tiers of diagnostic or therapeutic relevance; and compressing the plurality of regions includes using a level of compression respectively corresponding to each tier of the plurality of tiers of diagnostic or therapeutic relevance.

In some implementations, the method further includes prioritizing the plurality of regions into a sequence of ordered distinct image regions or specimen features based on the diagnostic or therapeutic relevance of each region of the plurality of regions; and wherein the metadata includes instructions for displaying the plurality of regions in an order based on the sequence.

In some implementations, the sequence of ordered distinct image regions is optimized based on one or more of: review efficiency; review thoroughness; directionality from one side to another side of the image; linear review of cell morphologies; and categorical review of cell morphologies.

In some implementations, the method further includes rendering the ordered distinct image regions on the display as a three-dimensional fly-through rendering of the image; wherein a first horizontal axis and a second horizontal axis of the three-dimensional fly-through rendering correspond to spatial components of the image, and a vertical axis of the three-dimensional fly-through rendering corresponds to the assigned levels of diagnostic or therapeutic relevance of each region of the image

In some implementations, the metadata includes parameter-based characterizations of cells, organelles, groups of cells or regions of cells, states of cells, or tissue morphologies of the specimen.

In some implementations, the metadata includes, for each region, a designation of a specialized generative adversarial network (GAN) model for subsequent reconstruction of the region.

In some implementations, the metadata includes, for each region, one or more instances from a library of specialized GAN models for subsequent reconstruction of the region (such GAN libraries may be comprised of hierarchical classes and various categories and degrees of specialization).

In some implementations, compressing the plurality of regions includes: de-resolving regions of the plurality of regions having diagnostic or therapeutic relevance under a threshold; and preserving an original resolution of regions of the plurality of regions having diagnostic or therapeutic relevance meeting the threshold.

In some implementations, de-resolving the regions having diagnostic or therapeutic relevance under the threshold includes de-resolving into fractionally pixel-shifted retrosource image layers for subsequent recombinant pixel-shift super-resolution at the first client device.

In some implementations, the method further includes, prior to receiving the request to view the image from the first client device: decompressing, using one or more specialized GANs, the plurality of regions into a plurality of reconstructed regions; comparing the plurality of reconstructed regions to pre-compressed versions of the plurality of regions; and based on the comparing, determining a difference between the reconstructed regions and the pre-compressed versions of the plurality of regions.

In some implementations, the method further includes, prior to receiving the request to view the image from the first client device: determining that the difference between the reconstructed regions and the pre-compressed versions of the plurality of regions meets a threshold; based on the determination that the difference between the reconstructed regions and the pre-compressed versions of the plurality of regions meets the threshold, updating the one or more specialized GANs; and re-compressing the plurality of regions using, for each region, a specialized GAN of the updated one or more specialized GANs; wherein transmitting the compressed plurality of regions includes transmitting the re-compressed plurality of regions.

In some implementations, the method further includes prior to receiving the request to view the image from the first client device: determining that the difference between the reconstructed regions and the pre-compressed versions of the plurality of regions does not meet the threshold; wherein transmitting the compressed plurality of regions is in accordance with the determination that the difference between the reconstructed regions and the pre-compressed versions of the plurality of regions does not meet the threshold.

In some implementations, the method further includes, at the server system: storing the compressed plurality of regions and the metadata; and prior to receiving the request to view the image from the first client device, deleting the image.

In some implementations, the method further includes, at the server system: packaging the compressed plurality of regions and the metadata into a file wrapper; wherein transmitting the compressed plurality of regions and the metadata to the first client device includes transmitting the file wrapper to the first client device.

In some implementations, the method further includes, at the first client device: receiving the compressed plurality of regions and the metadata from the server system; decompressing the compressed plurality of regions and the metadata; combining the decompressed regions into a reconstructed version of the image or a requested portion thereof; appending characteristic data included in the metadata corresponding to features of the specimen to corresponding regions of the reconstructed version of the image; and displaying portions of the reconstructed version of the image on a display integrated in or communicatively coupled to the first client device in an order based on the assigned levels of diagnostic or therapeutic relevance specified by the metadata.

In some implementations, the plurality of regions includes a first region having a first degree of diagnostic or therapeutic relevance and a second region having a second degree of diagnostic or therapeutic relevance lower than the first degree of diagnostic or therapeutic relevance; and compressing the plurality of regions includes compressing the first region using a first compression ratio of M:1 and compressing the second region using a second compression ratio of N:1, where N>M≥1.

In some implementations, the plurality of regions includes a first region having a first degree of diagnostic or therapeutic relevance and a second region having a second degree of diagnostic or therapeutic relevance lower than the first degree of diagnostic or therapeutic relevance; and compressing the plurality of regions includes compressing the first region using a lossless compression algorithm and compressing the second region using a lossy compression algorithm.

In some implementations, the plurality of regions includes a first region having a first degree of diagnostic or therapeutic relevance and a second region having a second degree of diagnostic or therapeutic relevance lower than the first degree of diagnostic or therapeutic relevance; and compressing the plurality of regions includes decreasing a resolution of the first region to an Mth degree and decreasing a resolution of the second region to an Nth degree, where N>M≥0.

In another aspect, a method of compressing and transmitting, reconstituting and presenting images for diagnostic annotation includes, at a server system including one or more processors: obtaining an image of a specimen (e.g., includes composite images derived from a volumetric z-stack, comprised of pixels, regions, or features selected for diagnostic or therapeutic relevance) (e.g., does not necessarily require a slide; tissue ribbon may be directly scanned without being mounted to a slide) (e.g., including specific circumstances associated with the protocols of one or more pharmaceutical trials and the matching of suitable participant candidates thereto); identifying one or more cellular morphologies of the specimen; mapping a plurality of regions of the image corresponding to the one or more cellular morphologies; assigning respective levels of diagnostic or therapeutic relevance to the plurality of regions; decreasing or maintaining respective resolutions of the plurality of regions based on the assigned levels of diagnostic or therapeutic relevance generating a plurality of processed regions; receiving a request to view the image from a first client device; and in response to receiving the request to view the image from the first client device, transmitting (i) the plurality of processed regions and (ii) metadata including an index of the assigned levels of diagnostic or therapeutic relevance of the plurality of processed regions to the first client device.

In some implementations, decreasing or maintaining respective resolutions of the plurality of regions based on the assigned levels of diagnostic or therapeutic relevance includes decreasing a resolution of at least one region of the plurality of regions, including reverse pixel shifting the at least one region.

In some implementations, reverse pixel shifting the at least one region includes: segmenting neighboring pixels of the image into a plurality of pixel groups; combining neighboring pixels of each pixel group of the plurality of pixel groups into a pixel group value (e.g., combining includes averaging or other mathematical function or algorithm, including neural network to anticipate and mitigate de-bayering artifacts or sensor noise); segmenting neighboring pixels of the image into a plurality of shifted pixel groups; averaging neighboring pixels of each shifted pixel group of the plurality of shifted pixel groups into a shifted pixel group value; and replacing the neighboring pixels of the image with a plurality of layers, including (i) a first layer comprising pixel group values of each pixel group and (ii) a second layer comprising shifted pixel group values of each shifted pixel group.

In some implementations, assigning respective levels of diagnostic or therapeutic relevance to the plurality of regions includes assigning a first degree of diagnostic or therapeutic relevance to a first region of the plurality of regions and assigning a second degree of diagnostic or therapeutic relevance lower than the first degree to a second region of the plurality of regions; and decreasing or maintaining respective resolutions of the plurality of regions based on the assigned levels of diagnostic or therapeutic relevance includes: decreasing a resolution of the first region to an Mth degree; and decreasing a resolution of the second region to an Nth degree, where N>M≥0.

In some implementations, assigning respective levels of diagnostic or therapeutic relevance to the plurality of regions includes assigning a first degree of diagnostic or therapeutic relevance to a first region of the plurality of regions and assigning a second degree of diagnostic or therapeutic relevance lower than the first degree to a second region of the plurality of regions; and decreasing or maintaining respective resolutions of the plurality of regions based on the assigned levels of diagnostic or therapeutic relevance includes: maintaining an original resolution of the first region based on a determination that a level of diagnostic or therapeutic relevance of the first region meets a threshold; and decreasing a resolution of the second region based on a determination that a level of diagnostic or therapeutic relevance of the second region does not meet the threshold.

In some implementations, decreasing or maintaining respective resolutions of the plurality of regions includes: de-resolving regions of the plurality of regions having diagnostic or therapeutic relevance under a threshold; and preserving an original resolution of regions of the plurality of regions having diagnostic or therapeutic relevance meeting the threshold.

In some implementations, de-resolving the regions having diagnostic or therapeutic relevance under the threshold includes de-resolving into fractionally pixel-shifted retrosource image layers for subsequent recombinant super-resolution at the first client device.

In some implementations, assigning the level of diagnostic or therapeutic relevance includes: submitting the image to one or more diagnostic machine vision systems (or human pathologist review); in response to submitting the image, receiving diagnostic or therapeutic relevance data associated with the plurality of regions from the one or more diagnostic machine vision systems; and aggregating the diagnostic or therapeutic relevance data received from the one or more diagnostic machine vision systems; wherein the assigning of the level of diagnostic or therapeutic relevance is based on the aggregated diagnostic or therapeutic relevance data received from the one or more diagnostic machine vision systems.

In some implementations, the method further includes extracting the plurality of regions into a plurality of discrete alpha layers or images, wherein decreasing or maintaining respective resolutions of the plurality of regions includes decreasing or maintaining respective resolutions of the plurality of discrete alpha layers or images; associating portions of the metadata with each of the plurality of discrete alpha layers or images; and respectively encoding or encrypting the portions of the metadata into the plurality of discrete alpha layers or images.

In some implementations, identifying the one or more cellular morphologies of the specimen includes compiling a cellular index of features of the image using a predefined library of tissue-specific or pathology-specific neural networks.

In some implementations, assigning the level of diagnostic or therapeutic relevance to each region includes assigning a plurality of tiers of diagnostic or therapeutic relevance; and decreasing or maintaining respective resolutions of the plurality of regions includes decreasing or maintaining respective resolutions using a degree of de-resolving respectively corresponding to each tier of the plurality of tiers of diagnostic or therapeutic relevance.

In some implementations, the method further includes prioritizing the plurality of regions into a sequence of ordered distinct image regions or specimen features based on the diagnostic or therapeutic relevance of each region of the plurality of regions; and wherein the metadata includes instructions for displaying the plurality of regions in an order based on the sequence.

In some implementations, the method further includes rendering the ordered distinct image regions on the display as a three-dimensional fly-through rendering of the image; wherein a first horizontal axis and a second horizontal axis of the three-dimensional fly-through rendering correspond to spatial components of the image, and a vertical axis of the three-dimensional fly-through rendering corresponds to the assigned levels of diagnostic or therapeutic relevance of each region of the image

In some implementations, the metadata includes parameter-based characterizations of cells, organelles, groups of cells or regions of cells, states of cells, or tissue morphologies of the specimen.

In some implementations, the metadata includes, for each region, a designation of a specialized generative adversarial network (GAN) model for subsequent reconstruction of the region.

In some implementations, the metadata includes, for each region, one or more instances from a library of specialized GAN models for subsequent reconstruction of the region (such GAN libraries may be comprised of hierarchical classes and various categories and degrees of specialization).

In some implementations, the method further includes, prior to receiving the request to view the image from the first client device: up-resolving, using one or more specialized GANs, the plurality of regions into a plurality of reconstructed regions; comparing the plurality of reconstructed regions to original versions of the plurality of regions; and based on the comparing, determining a difference between the reconstructed regions and the original versions of the plurality of regions.

In some implementations, the method further includes, prior to receiving the request to view the image from the first client device: determining that the difference between the reconstructed regions and the original versions of the plurality of regions meets a threshold; based on the determination that the difference between the reconstructed regions and the original versions of the plurality of regions meets the threshold, updating the one or more specialized GANs; and re-decreasing or maintaining respective resolutions of the plurality of regions using, for each region, a specialized GAN of the updated one or more specialized GANs; wherein transmitting the plurality of processed regions includes transmitting the plurality of regions with the re-decreased or maintained respective resolutions.

In some implementations, the method further includes, prior to receiving the request to view the image from the first client device: determining that the difference between the reconstructed regions and the original versions of the plurality of regions does not meet the threshold; wherein transmitting the plurality of processed regions is in accordance with the determination that the difference between the reconstructed regions and the original versions of the plurality of regions does not meet the threshold.

In some implementations, the method further includes, at the server system: storing the plurality of processed regions and the metadata; and prior to receiving the request to view the image from the first client device, deleting the image.

In some implementations, the method further includes, at the server system: packaging the plurality of processed regions and the metadata into a file wrapper; wherein transmitting the plurality of processed regions and the metadata to the first client device includes transmitting the file wrapper to the first client device.

In some implementations, the method further includes, at the first client device: receiving the plurality of processed regions and the metadata from the server system; up-resolving at least a subset of the plurality of processed regions and the metadata; combining the up-resolved regions into a reconstructed version of the image; appending characteristic data included in the metadata corresponding to features of the specimen to corresponding regions of the reconstructed version of the image; and displaying portions of the reconstructed version of the image on a display integrated in or communicatively coupled to the first client device in an order based on the assigned levels of diagnostic or therapeutic relevance specified by the metadata.

In some implementations, the method further includes compressing the plurality of regions using, for each region, a level of compression inversely related to the assigned level of diagnostic or therapeutic relevance for the region.

In some implementations, the plurality of regions includes a first region having a first degree of diagnostic or therapeutic relevance and a second region having a second degree of diagnostic or therapeutic relevance lower than the first degree of diagnostic or therapeutic relevance; and compressing the plurality of regions includes compressing the first region using a first compression ratio of M:1 and compressing the second region using a second compression ratio of N:1, where N>M≥1.

In some implementations, the plurality of regions includes a first region having a first degree of diagnostic or therapeutic relevance and a second region having a second degree of diagnostic or therapeutic relevance lower than the first degree of diagnostic or therapeutic relevance; and compressing the plurality of regions includes compressing the first region using a lossless compression algorithm and compressing the second region using a lossy compression algorithm.

In another aspect, a method of compressing and transmitting, reconstituting and presenting images for diagnostic annotation includes, at a server system including one or more processors: obtaining an image of a specimen (e.g., includes composite images derived from a volumetric z-stack, comprised of pixels, regions, or features selected for diagnostic or therapeutic relevance) (e.g., does not necessarily require a slide; tissue ribbon may be directly scanned without being mounted to a slide) (e.g., including specific circumstances associated with the protocols of one or more pharmaceutical trials and the matching of suitable participant candidates thereto); identifying one or more cellular morphologies of the specimen; mapping a plurality of regions of the image corresponding to the one or more cellular morphologies; compressing or de-resolving at least a subset of the plurality of regions into a plurality of compressed or de-resolved image segments; determining respective generative adversarial network (GAN) models that correspond to respective cellular morphologies associated with respective compressed or de-resolved image segments of the plurality of compressed or de-resolved image segments; and assigning the respective GAN models to the respective compressed or de-resolved image segments; receiving a request to view the image from a first client device; and in response to receiving the request to view the image from the first client device, transmitting (i) the plurality of compressed or de-resolved image segments, and (ii) the respective GAN models assigned to the plurality of compressed or de-resolved image segments to the first client device.

In some implementations, the method further includes, at the server system: constructing a map of the respective GAN models assigned to the plurality of compressed or de-resolved image segments, wherein segments of the map of the respective GAN models are linked to corresponding image segments of the plurality of compressed or de-resolved image segments; wherein transmitting the respective GAN models includes transmitting the map of the respective GAN models.

In some implementations, the method further includes, at the server system: compressing using a lossless compression algorithm or maintaining an original resolution of at least one region of the plurality of regions; forgoing determining and assigning a respective GAN model for the at least one region of the plurality of regions; and in response to receiving the request to view the image from the first client device, transmitting (iii) the at least one region compressed with the lossless compression algorithm or having the maintained original resolution to the first client device. algorithm.

In some implementations, the method further includes, at the server system: assigning respective levels of diagnostic or therapeutic relevance to the plurality of regions; determining that the at least one region of the plurality of regions meets a threshold of diagnostic or therapeutic relevance; determining that the subset of the plurality of regions does not meet the threshold of diagnostic or therapeutic relevance; wherein compressing using the lossless compression algorithm or maintaining the original resolution of the at least one region of the plurality of regions is in accordance with the determination that the at least one region of the plurality of regions meets the threshold of diagnostic or therapeutic relevance; and wherein compressing or de-resolving the subset of the plurality of regions and assigning the respective GAN models to the respective compressed or de-resolved image segments is in accordance with the determination that the subset of the plurality of regions does not meet the threshold of diagnostic or therapeutic relevance.

In some implementations, identifying the one or more cellular morphologies of the specimen includes compiling a cellular index of features of the image using a predefined library of tissue-specific or pathology-specific neural networks.

In some implementations, the compressing or the de-resolving includes de-resolving the subset of the plurality of regions into fractionally pixel-shifted retrosource image layers for subsequent recombinant pixel-shift super-resolution at the first client device.

In some implementations, the method further includes, prior to receiving the request to view the image from the first client device: decompressing or super-resolving, using the respective GAN models, the subset of regions into a plurality of reconstructed regions; comparing the plurality of reconstructed regions to pre-compressed or pre-de-resolved versions of the subset of regions; and based on the comparing, determining a difference between the reconstructed regions and the pre-compressed or pre-de-resolved versions of the subset of regions.

In some implementations, the method further includes, prior to receiving the request to view the image from the first client device: determining that the difference between the reconstructed regions and the pre-compressed or pre-de-resolved versions of the subset of regions meets a threshold; based on the determination that the difference between the reconstructed regions and the pre-compressed or pre-de-resolved versions of the subset of regions meets the threshold, updating the respective GAN models; and re-compressing or re-de-resolving the subset of the plurality of regions using the updated respective GAN models; wherein transmitting the plurality of compressed or de-resolved image segments includes transmitting the re-compressed or re-de-resolved subset of the plurality of regions.

In some implementations, the method further includes, prior to receiving the request to view the image from the first client device: determining that the difference between the reconstructed regions and the pre-compressed or pre-de-resolved versions of the subset of regions does not meet the threshold; wherein transmitting the plurality of compressed or de-resolved image segments is in accordance with the determination that the difference between the reconstructed regions and the pre-compressed or pre-de-resolved versions of the subset of regions does not meet the threshold.

In some implementations, the method further includes, at the server system: storing the plurality of compressed or de-resolved image segments and the respective GAN models assigned to the plurality of compressed or de-resolved image segments; and prior to receiving the request to view the image from the first client device, deleting the image.

In some implementations, the method further includes, at the server system: packaging the plurality of compressed or de-resolved image segments and the respective GAN models assigned to the plurality of compressed or de-resolved image segments into a file wrapper; wherein transmitting the plurality of compressed or de-resolved image segments and the respective GAN models assigned to the plurality of compressed or de-resolved image segments to the first client device includes transmitting the file wrapper to the first client device.

In some implementations, the method further includes, at the first client device: receiving the plurality of compressed or de-resolved image segments and the respective GAN models assigned to the plurality of compressed or de-resolved image segments from the server system; decompressing or super-resolving the compressed or de-resolved image segments using the respective GAN models assigned to the plurality of compressed or de-resolved image segments; combining the decompressed or super-resolved image segments into a reconstructed version of the image or a requested portion thereof; and displaying portions of the reconstructed version of the image on a display integrated in or communicatively coupled to the first client device.

In another aspect, a method of processing and transmitting images for diagnostic analysis includes, at a server system including one or more processors: obtaining an input image of a specimen; globally down-resolving the input image into a down-resolved image; subsequent to globally down-resolving the input image into the down-resolved image, concurrently: globally up-resolving the down-resolved image into an up-resolved image using a generative adversarial network (GAN) model configured to reconstruct images including features corresponding to the specimen; classifying a plurality of regions of the down-resolved image based on cellular morphologies and/or diagnostic relevance; and conveying the up-resolved image to a communication network for delivery to a client device.

In some implementations, the method further comprising dividing the input image into a plurality of tiles, wherein: globally down-resolving the input image includes down-resolving each of the plurality of tiles; and globally up-resolving the down-resolved image includes up-resolving each of the plurality of tiles.

In some implementations, globally up-resolving the down-resolved image includes using the GAN model to predictively improve clarity of the down-resolved image. In some implementations, globally up-resolving the down-resolved image includes restoring deleted pixels by predicting pixel values corresponding to the deleted pixels using the GAN model. In some implementations, globally up-resolving the down-resolved image includes overwriting de-resolved pixel values with pixel values predicted by the GAN model.

In some implementations, the method further comprises compressing the up-resolved image using a run-length encoding scheme prior to conveying the up-resolved image to the communication network.

In some implementations, the method further comprises manipulating a portion of the input image for subsequent processing based on the classifying of the plurality of regions. In some implementations, the subsequent processing includes re-globally down-resolving the input image having the manipulated portion, and concurrently globally up-resolving and classifying a plurality of regions of the re-globally down-resolved image.

In some implementations, a system comprises: one or more processors of a server or a client device and a memory storing instruction that, when executed by the one or more processors, cause the server or the client device to perform any of the methods described above.

In some implementations, a non-transitory computer readable storage medium stores instructions that, when executed by a server or a client device, cause the server or the client device to perform any of the methods described above.

In another aspect, a method of processing and transmitting images for diagnostic analysis comprises, at a server system including one or more processors: obtaining an input image of a specimen, wherein the input image includes image data representing a flattened z-stack; classifying spectral differences of a plurality of features of the input image; assigning z-levels of the z-stack to each of the plurality of features based on the classifying, including assigning one or more first z-levels to a first subset of the plurality of features (e.g., blood cells in a lower z-level) and one or more second z-levels to a second subset of the plurality of features (e.g., blood cells in a higher z-level), wherein the one or more first z-levels are underneath the one or more second z-levels thereby obscuring portions of the first subset of the plurality of features (e.g., a least a portion of a lower blood cell is obscured by at least a portion of a higher blood cell); predicting pixel values associated with the obscured portions of the first subset of the plurality of features using a generative adversarial network (GAN) model configured to reconstruct image features; generating three dimensional (3D) image data comprising the predicted pixel values and including image data from the one or more first z-levels and the one or more second z-levels, thereby representing a virtually reconstructed 3D z-stack; and providing the generated 3D image data for display on a client device.

In some implementations, generating the 3D image data includes: selecting a plurality of pixel values spanning a plurality of the z-levels and including at least a portion of the predicted pixel values that meet a predetermined threshold of sharpness; and replacing pixel values corresponding to obscured pixels with the selected pixel values.

In some implementations, generating the 3D image data includes: selecting a plurality of pixel values spanning a plurality of the z-levels and including at least a portion of the predicted pixel values that meet a predetermined threshold of diagnostic or therapeutic relevance; and replacing pixel values corresponding to obscured pixels with the selected pixel values.

In some implementations, classifying the spectral differences includes classifying borders of the features based on which spectral portions are most prevalent.

In some implementations, providing the generated 3D image data for display includes approximating navigation through a z-field including the z-stack by mapping a plurality of z-levels of the z-stack to respective control levels associated with a control user input element at the client device. In some implementations, the control user input element is a slider, a knob, a zoom control, or a z-field navigation control. In some implementations, approximating navigation through the z-stack is triggered after a zoom threshold has been met. In some implementations, generating the 3D image data includes generating a virtual slide or a non-planar virtual surface at an angle that bisects a plurality of the z-levels.

In some implementations, a system comprises: one or more processors of a server or a client device and a memory storing instruction that, when executed by the one or more processors, cause the server or the client device to perform any of the methods described above.

In some implementations, a non-transitory computer readable storage medium stores instructions that, when executed by a server or a client device, cause the server or the client device to perform any of the methods described above.

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims.

For example, specific features of the exemplary embodiments may or may not be part of the claimed invention, different components as opposed to those specifically mentioned may perform at least some of the features described herein, and features of the disclosed embodiments may be combined.

As used herein, the terms “about” and “approximately” may refer to + or −10% of the value referenced. For example, “about 9” is understood to encompass 8.2 and 9.9.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

It will be understood that, although the terms “first,” “second,” etc. are sometimes used herein to describe various elements, these elements should not be limited by these terms. These terms are only used to distinguish one element from another.

For example, a first element could be termed a second element, and, similarly, a second element could be termed a first element, without changing the meaning of the description, so long as all occurrences of the “first element” are renamed consistently and all occurrences of the second element are renamed consistently. The first element and the second element are both elements, but they are not the same element.

As used herein, the term “if” may be, optionally, construed to mean “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The terminology used herein is for the purpose of describing particular implementations only and is not intended to be limiting of the claims. For example, the image processing concepts described above can be used for non-medical images in addition to or as an alternative to the medical imaging examples described above. Any image data, regardless of its contents (medical or non-medical) can be processed by the image processing platform described herein using the same functions and modules.

As used in the description of the implementations and the appended claims, the singular forms “a”, “an” and “the” are intended to include the plural forms as well, unless the context clearly indicates otherwise.

It will also be understood that the term “and/or” as used herein refers to and encompasses any and all possible combinations of one or more of the associated listed items.

It will be further understood that the terms “comprises” and/or “comprising,” when used in this specification, specify the presence of stated features, integers, operations, elements, and/or components, but do not preclude the presence or addition of one or more other features, integers, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context.

Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

Further, to the extent that the method does not rely on the particular order of steps set forth herein, the particular order of the steps should not be construed as limitation on the claims. The claims directed to the method of the present invention should not be limited to the performance of their steps in the order written, and one skilled in the art can readily appreciate that the steps may be varied and still remain within the spirit and scope of the present invention.

1. A method of compressing, transmitting, reconstituting, and presenting images for diagnostic annotation, the method comprising:
at a server system including one or more processors:
obtaining an image of a specimen;
identifying one or more cellular morphologies of the specimen;
mapping a plurality of regions of the image corresponding to the one or more cellular morphologies;
assigning a level of diagnostic or therapeutic relevance to each region of the plurality of regions;
compressing the plurality of regions using, for each region, a level of compression inversely related to the assigned level of diagnostic or therapeutic relevance for the region;
receiving a request to view the image from a first client device; and
in response to receiving the request to view the image from the first client device, transmitting (i) the compressed plurality of regions and (ii) metadata including an index of the assigned levels of diagnostic or therapeutic relevance of the plurality of regions to the first client device.

2. The method of claim 1, wherein assigning the level of diagnostic or therapeutic relevance includes:
submitting the image to one or more diagnostic machine vision systems;
in response to submitting the image, receiving diagnostic or therapeutic relevance data associated with the plurality of regions from the one or more diagnostic machine vision systems; and
aggregating the diagnostic or therapeutic relevance data received from the one or more diagnostic machine vision systems;
wherein the assigning of the level of diagnostic or therapeutic relevance is based on the aggregated diagnostic or therapeutic relevance data received from the one or more diagnostic machine vision systems.

3. The method of any of the preceding claims, further comprising:
extracting the plurality of regions into a plurality of discrete alpha layers or images, wherein compressing the plurality of regions includes compressing the plurality of discrete alpha layers or images;
associating portions of the metadata with each of the plurality of discrete alpha layers or images; and
respectively encoding or encrypting the portions of the metadata into the plurality of discrete alpha layers or images.

4. The method of any of the preceding claims, wherein identifying the one or more cellular morphologies of the specimen includes compiling a cellular index of features of the image using a predefined library of tissue-specific or pathology-specific neural networks.

5. The method of any of the preceding claims, wherein:
assigning the level of diagnostic or therapeutic relevance to each region includes assigning a plurality of tiers of diagnostic or therapeutic relevance; and
compressing the plurality of regions includes using a level of compression respectively corresponding to each tier of the plurality of tiers of diagnostic or therapeutic relevance.

6. The method of any of the preceding claims, further comprising:
prioritizing the plurality of regions into a sequence of ordered distinct image regions or specimen features based on the diagnostic or therapeutic relevance of each region of the plurality of regions; and
wherein the metadata includes instructions for displaying the plurality of regions in an order based on the sequence.

7. The method of claim 6, wherein the sequence of ordered distinct image regions is optimized based on one or more of:
review efficiency;
review thoroughness;
directionality from one side to another side of the image;
linear review of cell morphologies; and
categorical review of cell morphologies.

8. The method of claim 6, further comprising:
rendering the ordered distinct image regions on the display as a three-dimensional fly-through rendering of the image;
wherein a first horizontal axis and a second horizontal axis of the three-dimensional fly-through rendering correspond to spatial components of the image, and a vertical axis of the three-dimensional fly-through rendering corresponds to the assigned levels of diagnostic or therapeutic relevance of each region of the image

9. The method of any of the preceding claims, wherein the metadata includes parameter-based characterizations of cells, organelles, groups of cells or regions of cells, states of cells, or tissue morphologies of the specimen.

10. The method of any of the preceding claims, wherein the metadata includes, for each region, a designation of a specialized generative adversarial network (GAN) model for subsequent reconstruction of the region.

11. The method of claim 10, wherein the metadata includes, for each region, one or more instances from a library of specialized GAN models for subsequent reconstruction of the region.

12. The method of any of the preceding claims, wherein compressing the plurality of regions includes:
de-resolving regions of the plurality of regions having diagnostic or therapeutic relevance under a threshold; and
preserving an original resolution of regions of the plurality of regions having diagnostic or therapeutic relevance meeting the threshold.

13. The method of claim 12, wherein de-resolving the regions having diagnostic or therapeutic relevance under the threshold includes de-resolving into fractionally pixel-shifted retrosource image layers for subsequent recombinant pixel-shift super-resolution at the first client device.

14. The method of any of the preceding claims, further comprising, prior to receiving the request to view the image from the first client device:
decompressing, using one or more specialized GANs, the plurality of regions into a plurality of reconstructed regions;
comparing the plurality of reconstructed regions to pre-compressed versions of the plurality of regions; and
based on the comparing, determining a difference between the reconstructed regions and the pre-compressed versions of the plurality of regions.

15. The method of claim 14, further comprising, prior to receiving the request to view the image from the first client device:
determining that the difference between the reconstructed regions and the pre-compressed versions of the plurality of regions meets a threshold;
based on the determination that the difference between the reconstructed regions and the pre-compressed versions of the plurality of regions meets the threshold, updating the one or more specialized GANs; and
re-compressing the plurality of regions using, for each region, a specialized GAN of the updated one or more specialized GANs;
wherein transmitting the compressed plurality of regions includes transmitting the re-compressed plurality of regions.

16. The method of claim 14, further comprising, prior to receiving the request to view the image from the first client device:
determining that the difference between the reconstructed regions and the pre-compressed versions of the plurality of regions does not meet the threshold;
wherein transmitting the compressed plurality of regions is in accordance with the determination that the difference between the reconstructed regions and the pre-compressed versions of the plurality of regions does not meet the threshold.

17. The method of any of the preceding claims, further comprising:
at the server system:
storing the compressed plurality of regions and the metadata; and
prior to receiving the request to view the image from the first client device, deleting the image.

18. The method of any of the preceding claims, further comprising:
at the server system:
packaging the compressed plurality of regions and the metadata into a file wrapper;
wherein transmitting the compressed plurality of regions and the metadata to the first client device includes transmitting the file wrapper to the first client device.

19. The method of any of the preceding claims, further comprising:
at the first client device:
receiving the compressed plurality of regions and the metadata from the server system;
decompressing the compressed plurality of regions and the metadata;
combining the decompressed regions into a reconstructed version of the image or a requested portion thereof;
appending characteristic data included in the metadata corresponding to features of the specimen to corresponding regions of the reconstructed version of the image; and
displaying portions of the reconstructed version of the image on a display integrated in or communicatively coupled to the first client device in an order based on the assigned levels of diagnostic or therapeutic relevance specified by the metadata.

20. The method of any of the preceding claims, wherein:
the plurality of regions includes a first region having a first degree of diagnostic or therapeutic relevance and a second region having a second degree of diagnostic or therapeutic relevance lower than the first degree of diagnostic or therapeutic relevance; and
compressing the plurality of regions includes compressing the first region using a first compression ratio of M:1 and compressing the second region using a second compression ratio of N:1, where N>M≥1.

21. The method of any of the preceding claims, wherein:
the plurality of regions includes a first region having a first degree of diagnostic or therapeutic relevance and a second region having a second degree of diagnostic or therapeutic relevance lower than the first degree of diagnostic or therapeutic relevance; and
compressing the plurality of regions includes compressing the first region using a lossless compression algorithm and compressing the second region using a lossy compression algorithm.

22. The method of any of the preceding claims, wherein:
the plurality of regions includes a first region having a first degree of diagnostic or therapeutic relevance and a second region having a second degree of diagnostic or therapeutic relevance lower than the first degree of diagnostic or therapeutic relevance; and
compressing the plurality of regions includes decreasing a resolution of the first region to an Mth degree and decreasing a resolution of the second region to an Nth degree, where N>M≥0.

23. A system comprising: one or more processors of a server or a client device and a memory storing instruction that, when executed by the one or more processors, cause the server or the client device to perform any of the methods of claims 1-22.

24. A non-transitory computer readable storage medium storing instructions that, when executed by a server or a client device, cause the server or the client device to perform any of the methods of claims 1-22.

25. A method of compressing and transmitting, reconstituting and presenting images for diagnostic annotation, the method comprising:
at a server system including one or more processors:
obtaining an image of a specimen;
identifying one or more cellular morphologies of the specimen;
mapping a plurality of regions of the image corresponding to the one or more cellular morphologies;
assigning respective levels of diagnostic or therapeutic relevance to the plurality of regions;
decreasing or maintaining respective resolutions of the plurality of regions based on the assigned levels of diagnostic or therapeutic relevance generating a plurality of processed regions;
receiving a request to view the image from a first client device; and
in response to receiving the request to view the image from the first client device, transmitting (i) the plurality of processed regions and (ii) metadata including an index of the assigned levels of diagnostic or therapeutic relevance of the plurality of processed regions to the first client device.

26. The method of claim 25, wherein:
decreasing or maintaining respective resolutions of the plurality of regions based on the assigned levels of diagnostic or therapeutic relevance includes decreasing a resolution of at least one region of the plurality of regions, including reverse pixel shifting the at least one region.

27. The method of claim 26, wherein reverse pixel shifting the at least one region includes:
segmenting neighboring pixels of the image into a plurality of pixel groups;
combining neighboring pixels of each pixel group of the plurality of pixel groups into a pixel group value;
segmenting neighboring pixels of the image into a plurality of shifted pixel groups;
averaging neighboring pixels of each shifted pixel group of the plurality of shifted pixel groups into a shifted pixel group value; and
replacing the neighboring pixels of the image with a plurality of layers, including (i) a first layer comprising pixel group values of each pixel group and (ii) a second layer comprising shifted pixel group values of each shifted pixel group.

28. The method of any of the preceding claims, wherein:
assigning respective levels of diagnostic or therapeutic relevance to the plurality of regions includes assigning a first degree of diagnostic or therapeutic relevance to a first region of the plurality of regions and assigning a second degree of diagnostic or therapeutic relevance lower than the first degree to a second region of the plurality of regions; and
decreasing or maintaining respective resolutions of the plurality of regions based on the assigned levels of diagnostic or therapeutic relevance includes:
decreasing a resolution of the first region to an Mth degree; and
decreasing a resolution of the second region to an Nth degree, where N>M≥0.

29. The method of any of the preceding claims, wherein:
assigning respective levels of diagnostic or therapeutic relevance to the plurality of regions includes assigning a first degree of diagnostic or therapeutic relevance to a first region of the plurality of regions and assigning a second degree of diagnostic or therapeutic relevance lower than the first degree to a second region of the plurality of regions; and
decreasing or maintaining respective resolutions of the plurality of regions based on the assigned levels of diagnostic or therapeutic relevance includes:
maintaining an original resolution of the first region based on a determination that a level of diagnostic or therapeutic relevance of the first region meets a threshold; and
decreasing a resolution of the second region based on a determination that a level of diagnostic or therapeutic relevance of the second region does not meet the threshold.

30. The method of any of the preceding claims, wherein decreasing or maintaining respective resolutions of the plurality of regions includes:
de-resolving regions of the plurality of regions having diagnostic or therapeutic relevance under a threshold; and
preserving an original resolution of regions of the plurality of regions having diagnostic or therapeutic relevance meeting the threshold.

31. The method of claim 30, wherein de-resolving the regions having diagnostic or therapeutic relevance under the threshold includes de-resolving into fractionally pixel-shifted retrosource image layers for subsequent recombinant super-resolution at the first client device.

32. The method of any of the preceding claims, wherein assigning the level of diagnostic or therapeutic relevance includes:
submitting the image to one or more diagnostic machine vision systems;
in response to submitting the image, receiving diagnostic or therapeutic relevance data associated with the plurality of regions from the one or more diagnostic machine vision systems; and
aggregating the diagnostic or therapeutic relevance data received from the one or more diagnostic machine vision systems;
wherein the assigning of the level of diagnostic or therapeutic relevance is based on the aggregated diagnostic or therapeutic relevance data received from the one or more diagnostic machine vision systems.

33. The method of any of the preceding claims, further comprising:
extracting the plurality of regions into a plurality of discrete alpha layers or images, wherein decreasing or maintaining respective resolutions of the plurality of regions includes decreasing or maintaining respective resolutions of the plurality of discrete alpha layers or images;
associating portions of the metadata with each of the plurality of discrete alpha layers or images; and
respectively encoding or encrypting the portions of the metadata into the plurality of discrete alpha layers or images.

34. The method of any of the preceding claims, wherein identifying the one or more cellular morphologies of the specimen includes compiling a cellular index of features of the image using a predefined library of tissue-specific or pathology-specific neural networks.

35. The method of any of the preceding claims, wherein:
assigning the level of diagnostic or therapeutic relevance to each region includes assigning a plurality of tiers of diagnostic or therapeutic relevance; and
decreasing or maintaining respective resolutions of the plurality of regions includes decreasing or maintaining respective resolutions using a degree of de-resolving respectively corresponding to each tier of the plurality of tiers of diagnostic or therapeutic relevance.

36. The method of any of the preceding claims, further comprising:
prioritizing the plurality of regions into a sequence of ordered distinct image regions or specimen features based on the diagnostic or therapeutic relevance of each region of the plurality of regions; and
wherein the metadata includes instructions for displaying the plurality of regions in an order based on the sequence.

37. The method of claim 36, further comprising:
rendering the ordered distinct image regions on the display as a three-dimensional fly-through rendering of the image;
wherein a first horizontal axis and a second horizontal axis of the three-dimensional fly-through rendering correspond to spatial components of the image, and a vertical axis of the three-dimensional fly-through rendering corresponds to the assigned levels of diagnostic or therapeutic relevance of each region of the image

38. The method of any of the preceding claims, wherein the metadata includes parameter-based characterizations of cells, organelles, groups of cells or regions of cells, states of cells, or tissue morphologies of the specimen.

39. The method of any of the preceding claims, wherein the metadata includes, for each region, a designation of a specialized generative adversarial network (GAN) model for subsequent reconstruction of the region.

40. The method of claim 39, wherein the metadata includes, for each region, one or more instances from a library of specialized GAN models for subsequent reconstruction of the region.

41. The method of any of the preceding claims, further comprising, prior to receiving the request to view the image from the first client device:
up-resolving, using one or more specialized GANs, the plurality of regions into a plurality of reconstructed regions;
comparing the plurality of reconstructed regions to original versions of the plurality of regions; and
based on the comparing, determining a difference between the reconstructed regions and the original versions of the plurality of regions.

42. The method of claim 41, further comprising, prior to receiving the request to view the image from the first client device:
determining that the difference between the reconstructed regions and the original versions of the plurality of regions meets a threshold;
based on the determination that the difference between the reconstructed regions and the original versions of the plurality of regions meets the threshold, updating the one or more specialized GANs; and
re-decreasing or maintaining respective resolutions of the plurality of regions using, for each region, a specialized GAN of the updated one or more specialized GANs;
wherein transmitting the plurality of processed regions includes transmitting the plurality of regions with the re-decreased or maintained respective resolutions.

43. The method of claim 41, further comprising, prior to receiving the request to view the image from the first client device:
determining that the difference between the reconstructed regions and the original versions of the plurality of regions does not meet the threshold;
wherein transmitting the plurality of processed regions is in accordance with the determination that the difference between the reconstructed regions and the original versions of the plurality of regions does not meet the threshold.

44. The method of any of the preceding claims, further comprising:
at the server system:
storing the plurality of processed regions and the metadata; and
prior to receiving the request to view the image from the first client device, deleting the image.

45. The method of any of the preceding claims, further comprising:
at the server system:
packaging the plurality of processed regions and the metadata into a file wrapper;
wherein transmitting the plurality of processed regions and the metadata to the first client device includes transmitting the file wrapper to the first client device.

46. The method of any of the preceding claims, further comprising:
at the first client device:
receiving the plurality of processed regions and the metadata from the server system;
up-resolving at least a subset of the plurality of processed regions and the metadata;
combining the up-resolved regions into a reconstructed version of the image;
appending characteristic data included in the metadata corresponding to features of the specimen to corresponding regions of the reconstructed version of the image; and
displaying portions of the reconstructed version of the image on a display integrated in or communicatively coupled to the first client device in an order based on the assigned levels of diagnostic or therapeutic relevance specified by the metadata.

47. The method of any of the preceding claims, further comprising:
compressing the plurality of regions using, for each region, a level of compression inversely related to the assigned level of diagnostic or therapeutic relevance for the region.

48. The method of claim 47, wherein:
the plurality of regions includes a first region having a first degree of diagnostic or therapeutic relevance and a second region having a second degree of diagnostic or therapeutic relevance lower than the first degree of diagnostic or therapeutic relevance; and
compressing the plurality of regions includes compressing the first region using a first compression ratio of M:1 and compressing the second region using a second compression ratio of N:1, where N>M≥1.

49. The method of claim 47, wherein:
the plurality of regions includes a first region having a first degree of diagnostic or therapeutic relevance and a second region having a second degree of diagnostic or therapeutic relevance lower than the first degree of diagnostic or therapeutic relevance; and
compressing the plurality of regions includes compressing the first region using a lossless compression algorithm and compressing the second region using a lossy compression algorithm.

50. A system comprising: one or more processors of a server or a client device and a memory storing instruction that, when executed by the one or more processors, cause the server or the client device to perform any of the methods of claims 25-49.

51. A non-transitory computer readable storage medium storing instructions that, when executed by a server or a client device, cause the server or the client device to perform any of the methods of claims 25-49.

52. A method of compressing and transmitting, reconstituting and presenting images for diagnostic annotation, the method comprising:
at a server system including one or more processors:
obtaining an image of a specimen;
identifying one or more cellular morphologies of the specimen;
mapping a plurality of regions of the image corresponding to the one or more cellular morphologies;
compressing or de-resolving at least a subset of the plurality of regions into a plurality of compressed or de-resolved image segments;
determining respective generative adversarial network (GAN) models that correspond to respective cellular morphologies associated with respective compressed or de-resolved image segments of the plurality of compressed or de-resolved image segments; and
assigning the respective GAN models to the respective compressed or de-resolved image segments;
receiving a request to view the image from a first client device; and
in response to receiving the request to view the image from the first client device, transmitting (i) the plurality of compressed or de-resolved image segments, and (ii) the respective GAN models assigned to the plurality of compressed or de-resolved image segments to the first client device.

53. The method of any of the preceding claims, further comprising:
at the server system:
constructing a map of the respective GAN models assigned to the plurality of compressed or de-resolved image segments, wherein segments of the map of the respective GAN models are linked to corresponding image segments of the plurality of compressed or de-resolved image segments;
wherein transmitting the respective GAN models includes transmitting the map of the respective GAN models.

54. The method of any of the preceding claims, further comprising:
at the server system:
compressing using a lossless compression algorithm or maintaining an original resolution of at least one region of the plurality of regions;
forgoing determining and assigning a respective GAN model for the at least one region of the plurality of regions; and
in response to receiving the request to view the image from the first client device, transmitting (iii) the at least one region compressed with the lossless compression algorithm or having the maintained original resolution to the first client device. algorithm.

55. The method of claim 54, further comprising:
at the server system:
assigning respective levels of diagnostic or therapeutic relevance to the plurality of regions;
determining that the at least one region of the plurality of regions meets a threshold of diagnostic or therapeutic relevance;
determining that the subset of the plurality of regions does not meet the threshold of diagnostic or therapeutic relevance;
wherein compressing using the lossless compression algorithm or maintaining the original resolution of the at least one region of the plurality of regions is in accordance with the determination that the at least one region of the plurality of regions meets the threshold of diagnostic or therapeutic relevance; and
wherein compressing or de-resolving the subset of the plurality of regions and assigning the respective GAN models to the respective compressed or de-resolved image segments is in accordance with the determination that the subset of the plurality of regions does not meet the threshold of diagnostic or therapeutic relevance.

56. The method of any of the preceding claims, wherein identifying the one or more cellular morphologies of the specimen includes compiling a cellular index of features of the image using a predefined library of tissue-specific or pathology-specific neural networks.

57. The method of any of the preceding claims, wherein the compressing or the de-resolving includes de-resolving the subset of the plurality of regions into fractionally pixel-shifted retrosource image layers for subsequent recombinant pixel-shift super-resolution at the first client device.

58. The method of any of the preceding claims, further comprising, prior to receiving the request to view the image from the first client device:
decompressing or super-resolving, using the respective GAN models, the subset of regions into a plurality of reconstructed regions;
comparing the plurality of reconstructed regions to pre-compressed or pre-de-resolved versions of the subset of regions; and
based on the comparing, determining a difference between the reconstructed regions and the pre-compressed or pre-de-resolved versions of the subset of regions.

59. The method of claim 58, further comprising, prior to receiving the request to view the image from the first client device:
determining that the difference between the reconstructed regions and the pre-compressed or pre-de-resolved versions of the subset of regions meets a threshold;
based on the determination that the difference between the reconstructed regions and the pre-compressed or pre-de-resolved versions of the subset of regions meets the threshold, updating the respective GAN models; and
re-compressing or re-de-resolving the subset of the plurality of regions using the updated respective GAN models;
wherein transmitting the plurality of compressed or de-resolved image segments includes transmitting the re-compressed or re-de-resolved subset of the plurality of regions.

60. The method of claim 58, further comprising, prior to receiving the request to view the image from the first client device:
determining that the difference between the reconstructed regions and the pre-compressed or pre-de-resolved versions of the subset of regions does not meet the threshold;
wherein transmitting the plurality of compressed or de-resolved image segments is in accordance with the determination that the difference between the reconstructed regions and the pre-compressed or pre-de-resolved versions of the subset of regions does not meet the threshold.

61. The method of any of the preceding claims, further comprising:
at the server system:
storing the plurality of compressed or de-resolved image segments and the respective GAN models assigned to the plurality of compressed or de-resolved image segments; and
prior to receiving the request to view the image from the first client device, deleting the image.

62. The method of any of the preceding claims, further comprising:
at the server system:
packaging the plurality of compressed or de-resolved image segments and the respective GAN models assigned to the plurality of compressed or de-resolved image segments into a file wrapper;
wherein transmitting the plurality of compressed or de-resolved image segments and the respective GAN models assigned to the plurality of compressed or de-resolved image segments to the first client device includes transmitting the file wrapper to the first client device.

63. The method of any of the preceding claims, further comprising:
at the first client device:
receiving the plurality of compressed or de-resolved image segments and the respective GAN models assigned to the plurality of compressed or de-resolved image segments from the server system;
decompressing or super-resolving the compressed or de-resolved image segments using the respective GAN models assigned to the plurality of compressed or de-resolved image segments;
combining the decompressed or super-resolved image segments into a reconstructed version of the image or a requested portion thereof; and
displaying portions of the reconstructed version of the image on a display integrated in or communicatively coupled to the first client device.

64. A system comprising: one or more processors of a server or a client device and a memory storing instruction that, when executed by the one or more processors, cause the server or the client device to perform any of the methods of claims 50-63.

65. A non-transitory computer readable storage medium storing instructions that, when executed by a server or a client device, cause the server or the client device to perform any of the methods of claims 52-63.

66. A method of processing and transmitting images for diagnostic analysis, the method comprising:
at a server system including one or more processors:
obtaining an input image of a specimen;
globally down-resolving the input image into a down-resolved image;
subsequent to globally down-resolving the input image into the down-resolved image, concurrently:
globally up-resolving the down-resolved image into an up-resolved image using a generative adversarial network (GAN) model configured to reconstruct images including features corresponding to the specimen; and
classifying a plurality of regions of the down-resolved image based on cellular morphologies and/or diagnostic relevance; and
conveying the up-resolved image to a communication network for delivery to a client device.

67. The method of any of the preceding claims, further comprising dividing the input image into a plurality of tiles, wherein:
globally down-resolving the input image includes down-resolving each of the plurality of tiles; and
globally up-resolving the down-resolved image includes up-resolving each of the plurality of tiles.

68. The method of any of the preceding claims, wherein globally up-resolving the down-resolved image includes using the GAN model to predictively improve clarity of the down-resolved image.

69. The method of any of the preceding claims, wherein globally up-resolving the down-resolved image includes restoring deleted pixels by predicting pixel values corresponding to the deleted pixels using the GAN model.

70. The method of any of the preceding claims, wherein globally up-resolving the down-resolved image includes overwriting de-resolved pixel values with pixel values predicted by the GAN model.

71. The method of any of the preceding claims, further comprising compressing the up-resolved image using a run-length encoding scheme prior to conveying the up-resolved image to the communication network.

72. The method of any of the preceding claims, further comprising manipulating a portion of the input image for subsequent processing based on the classifying of the plurality of regions.

73. The method of claim 72, wherein the subsequent processing includes re-globally down-resolving the input image having the manipulated portion, and concurrently globally up-resolving and classifying a plurality of regions of the re-globally down-resolved image.

74. A system comprising: one or more processors of a server or a client device and a memory storing instruction that, when executed by the one or more processors, cause the server or the client device to perform any of the methods of claims 66-73.

75. A non-transitory computer readable storage medium storing instructions that, when executed by a server or a client device, cause the server or the client device to perform any of the methods of claims 66-73.

76. A method of processing and transmitting images for diagnostic analysis, the method comprising:
at a server system including one or more processors:
obtaining an input image of a specimen, wherein the input image includes image data representing a flattened z-stack;
classifying spectral differences of a plurality of features of the input image;
assigning z-levels of the z-stack to each of the plurality of features based on the classifying, including assigning one or more first z-levels to a first subset of the plurality of features and one or more second z-levels to a second subset of the plurality of features, wherein the one or more first z-levels are underneath the one or more second z-levels thereby obscuring portions of the first subset of the plurality of features;
predicting pixel values associated with the obscured portions of the first subset of the plurality of features using a generative adversarial network (GAN) model configured to reconstruct image features;
generating three dimensional (3D) image data comprising the predicted pixel values and including image data from the one or more first z-levels and the one or more second z-levels, thereby representing a virtually reconstructed 3D z-stack; and
providing the generated 3D image data for display on a client device.

77. The method of any of the preceding claims, wherein generating the 3D image data includes:
selecting a plurality of pixel values spanning a plurality of the z-levels and including at least a portion of the predicted pixel values that meet a predetermined threshold of sharpness; and
replacing pixel values corresponding to obscured pixels with the selected pixel values.

78. The method of any of the preceding claims, wherein generating the 3D image data includes:
selecting a plurality of pixel values spanning a plurality of the z-levels and including at least a portion of the predicted pixel values that meet a predetermined threshold of diagnostic or therapeutic relevance; and
replacing pixel values corresponding to obscured pixels with the selected pixel values.

79. The method of any of the preceding claims, wherein classifying the spectral differences includes classifying borders of the features based on which spectral portions are most prevalent.

80. The method of any of the preceding claims, wherein providing the generated 3D image data for display includes approximating navigation through a z-field including the z-stack by mapping a plurality of z-levels of the z-stack to respective control levels associated with a control user input element at the client device.

81. The method of claim 80, wherein the control user input element is a slider, a knob, a zoom control, or a z-field navigation control.

82. The method of claim 80, wherein approximating navigation through the z-stack is triggered after a zoom threshold has been met.

83. The method of any of the preceding claims, wherein generating the 3D image data includes generating a virtual slide or a non-planar virtual surface at an angle that bisects a plurality of the z-levels.

84. A system comprising: one or more processors of a server or a client device and a memory storing instruction that, when executed by the one or more processors, cause the server or the client device to perform any of the methods of claims 76-83.

85. A non-transitory computer readable storage medium storing instructions that, when executed by a server or a client device, cause the server or the client device to perform any of the methods of claims 76-83.
